SEQUENCE LISTING (27 sequences)

SEQ ID NO: 1 - DNA, 17572 bp, Bombyx mori
atgagagtcaaaacctttgtgatcttgtgctgcgctctgcaggtgagttaattattttactattatttcagaaggtggccagacgatatcacgggccacctgataataagtggtcgccaaaacgcacagatatcgtaaattgtgccatttgatttgtcacgcccgggggggctacggaataaactacatttatttatttaaaaaatgaaccttagattatgtaacttgtgatttatttgcgtcaaaagtaggcaagatgaatctatgtaaatacctgggcagacttgcaatatcctatttcaccggtaaatcagcattgcaatatgcaatgcatattcaacaatatgtaaaacaattcgtaaagcatcattagaaaatagacgaaagaaattgcataaaattataaccgcattattaatttattatgatatctattaacaattgctattgcctttttttcgcaaattataatcattttcataacctcgaggtagcattctgttacattttaatacattggtatgtgattataacacgagctgcccactgagtttctcgccagatcttctcagtgggtcgcgttaccgatcacgtgatagattctatgaagcactgctcttgttagggctagtgttagcaaattctttcaggttgagtctgagagctcacctacccatcggagcgtagctggaataggctaccagctaataggtagggaaaacaaagctcgaaacaagctcaagtaataacaacataatgtgaccataaaatctcgtggtgtatgagatacaattatgtactttcccacaaatgtttacataattagaatgttgttcaacttgcctaacgccccagctagaacattcaattattactattaccactactaaggcagtatgtcctaactcgttccagatcagcgctaacttcgattgaatgtgcgaaatttatagctcaatattttagcacttatcgtattgatttaagaaaaaattgttaacattttgtttcagtatgtcgcttatacaaatgcaaacatcaatgattttgatgaggactattttgggagtgatgtcactgtccaaagtagtaatacaacagatgaaataattagagatgcatctggggcagttatcgaagaacaaattacaactaaaaaaatgcaacggaaaaataaaaaccatggaatacttggaaaaaatgaaaaaatgatcaagacgttcgttataaccacggattccgacggtaacgagtccattgtagaggaagatgtgctcatgaagacactttccgatggtactgttgctcaaagttatgttgctgctgatgcgggagcatattctcagagcgggccatacgtatcaaacagtggatacagcactcatcaaggatatacgagcgatttcagcactagtgctgcagtcggtgcaggagctggtgcaggtgctgccgctggttctggtgcgggtgccggagctggttatggagctgcttctggtgctggtgccggtgctggggctggtgccggagctggttatggaactggtgcaggtgcaggtgccggagctggttatggagctggtgcaggtgcaggtgccggagctggttatggggctggtgcaggtgcaggtgccggagctggttatggagctggtgcaggtgcaggtgccggagctggttatggggctggtgcaggtgcaggtgccggagctggttatggagctggtgcgggtgccggtgccggggctggttatggagctgcctctggtgctggtgctggcgctgggtacggacaaggagtaggaagcggagctgcttctggagctggtgcaggtgcaggagcaggttctgccgctggttctggggcaggtgccggtgctggtaccggtgctggtgcaggttacggagctggtgcaggtgccggtgccggagctggttatggagctgcctctggtactggagcaggttatggagctggtgccggagctggttacggaggtgcctctggtgctggtgctggtgccggtgctggggctggagccggtgctggtgcaggttatggaactggcgctggatacggagcaggagccggagcaggagccggagcaggagctggtgctggatacggagcaggagctggtgctggatacggagcaggatatggagtaggagctggtgctggatacggagcaggatacggagcaggagctggaagcggagctgcctctggtgctggttcaggtgccggtgctggttcaggtgccggtgctggttcaggtgccggtgctggttcaggtgccggtgccggttcaggtgctggtgctggttcaggtgctggtgctggttcaggtgctggtgcaggttcaggtgctggtgctggttcaggtactggtgctggttcaggagctggtgctggatacggagcaggagctggtgctggatacggagcaggagcaggaagtggagctgcctctggtgccggtgctggttcaggtgcaggtgctggttcaggtgctggtgctggttcaggtgctggtgctggttcaggtgctggtgctggttcaggagctggtgctggatacggagcaggagctggtgctggatacggagcaggagctggtgctggatacggagcaggagctggcgttggatacggagcaggagctggaagcggagctgcctctggtgctggtgctggttcaggagccggtgctggttcaggtgctggtgctggttcaggtgctggtgctggttcaggtgctggtgctggttcaggtgccggtgctggttcaggtgctggtgctggttcaggagctggtgctggttcaggtgctggtgctggttcaggagctggagttggatacggagcaggagttggtgctggatacggagcaggatatggagcaggagctggtgctggatacggagcaggagcaggaagcggagctgcctctggtgctggtgccggtgctggagctggtgcaggaacaggctcttctggatttggaccatatgtagcaaatggcggatatagcagaagtgatggctacgaatacgcttggtcgtctgactttggaactggaagcggagctggtgctggttcaggtgctggtgctggttcaggtgctggcgctggctcaggtgctggtgctggttcaggtgctggtgctggttcaggagctggagctggatacggagcaggagttggtgttggatacggagcaggatatggagcaggagctggtgctggatacggagcaggagcaggaagcggagctgcctctggtgccggtgctggttcaggtgctggtgctggttcaggtgccggtgctggttcaggtgctggtgctggttcaggtgccggtgctggttcaggtgctggtgctggctcaggtgccggtgctggttcaggtgctggtgctggttcaggtgccggtgctggttcaggtgctggtgctggttcaggtgctggtgttggctcaggtgctggtgctggttcaggtgctggtgctggcgttggatacggagcaggagctggcgttggatacggagcaggagctggaagcggagctgcctctggtgctggtgctggttcaggagccggtgctggttcaggtgctggtgctggttcaggtgctggtgctggttcaggtgctggtgctggttcaggtgccggtgctggttcaggtgctggtgctggttcaggagctggtgctggttcaggtgctggtgctggttcaggagctggagttggatacggagcaggagttggtgctggatacggagcaggatatggagcaggagctggtgctggatacggagcaggagcaggaagcggagctgcctcaggtgccggtgctggttcaggtgctggtgctggttcaggtgccggtgctggttcaggtgctggtgctggttcaggtgctggtgctggttcaggtgctggtgctggttcaggtgctggtgctggttcaggtgctggtgctggttcaggtgctggtgctggctcaggtgctggagctggttcaggtgctggtgctggttcaggagctggagctggatacggagcaggagctggtgctggatacggagcaggatatggagcaggagctggtgctggatacggagcaggagctggaagcggagctgcctctggtgctggttcaggtgccggtgctggttcaggtgccggtgctggtgctggttcaggtgctggtgctggttcaggtgctggtgctggttcaggtgctggtgctggctcaggtgctggagctggttcaggtgctggtgctggttcaggagctggagctggatacggagcaggagttggtgctggatacggagcaggatatggagcaggagctggtgctggatacggagcaggagcaggaagcggagctgcctctggtgccggtgctggttcaggtgctggagctggttcaggtgccggtgctggttcaggtgctggtgctggttcaggagctggtgctggttcaggagctggtgctggttcaggtgctggtgctggttcaggagctggagttggatacggagcaggatatggagcaggagctggtgctggatacggagcaggagcaggaagcggagctgcctctggtgctggtgccggtgctggagctggtgcaggaacaggctcttctggatttggaccatatgtagcacatggcggatatagcggctacgaatacgcttggtcgtcagaatctgactttggaactggaagcggagctggtgctggttcaggtgctggtgctggttcaggtgctggtgctggctcaggtgctggtgctggttcaggagctggatacggagcaggagttggtgctggatacggagcaggatatggagcaggagctggtgctggatacggagcaggagcaggaagcggagctggctcaggtgctggtgctggttcaggagctggagctggttcaggtgccggtgctggttcaggtgctggtgctggttcaggagctggtgctggttcaggtgctggtgctggttcaggagctggtgctggttcaggtgccggtgctggttcaggtgctggtgctggatacggagcaggatatggagcaggagctggtgctggatacggagcaggagcaggaagcggagctggctcaggtgctggtgctggttcaggtgctggtgctggttcaggagctggagctggttcaggtgccggtgctggttcaggtgctggtgctggttcaggtgctggtgctggctcaggtgctggtgctggttcaggagctggagctggatacggagcaggagttggtgctggatacggagcaggatatggagcaggagctggtgctggatacggagcaggagcaggaagcggagctggctcaggtgctggtgctggttcaggagctggagctggttcaggtgctggtgctggttcaggtgccggtgttggttcaggtgctggtgctggttcaggagctggtgctggttcaggtgccggtgctggttcaggtgctggtgctggatacggagcaggatatggagcaggagctggtgctggatacggagcaggagcaggaagcggagctggctcaggtgctggtgctggttcaggtgctggtgctggttcaggagctggagctggttcaggtgccggtgctggttcaggtgctggtgctggttcaggagctggtgctggttcaggtgctggtgctggttcaggagctggagttggatacggagcaggagttggtgctggatacggagcaggatatggagcaggagctggtgctggatacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggttcaggtgctggtgctggttcaggtgctggtgctggctcaggtgctggtgctggttcaggagctggagctggatacggagcaggatatggagcaggagctggtgctggatacggagcaggagcaggaagcggagctggctcaggtgctggtgctggttcaggagctggagctggttcaggtgccggtgctggttcaggtgctggtgctggttcaggtgccggtgctggttcaggtgctggtgctggttcaggagctggtgctggttcaggtgccggttctggttcaggagctggtgctggttcaggtgctggtgctggttcaggagctggagctggatacggagcaggagttggtgctggatacggagtaggatatggagcaggagctggtgctggatacggagcaggagcaggaagcggagctgcctctggtgctggtgccggtgctggagctggtgcaggaacaggctcttctggatttggaccatatgtagcacatggcggatatagcggctacgaatacgcttggtcgtcagaatctgactttggaactggaagcggagctggtgctggttcaggtgctggtgctggttcaggtgctggtgctggctcaggtgctggtgctggttcaggagctggtgctggttcaggtgctggtgctggttcaggagctggagctggatacggagcaggagttggtgctggatacggagcagcatatggagcaggagctggtgctggatacggagctggagcaggaagcggagctgcctctggtgccggtgctggttcaggtgctggtgctggttcaggtgccggtgctggttcaggtgctggtgctggttcaggtgctggtgctggttcaggtgctggtgctggttcaggtgctggtgctggttcaggagctggtgctggttcaggagctggtgctggttcaggtgccggtgctggttcaggagctggagcaggatatggagcaggagctggtgctggatacggagcaggagcaggaagcggagctggctcaggtgctggtgctggttcaggtgctggtgctggttcaggagctggagctggttcaggtgccggtgctggttcaggtgctggtgctggttcaggtgccggttctggttcaggagctggtgctggttcaggtgctggtgctggttcaggagctggagctggatacggagcaggagttggtgctggatacggagcaggatatggagcaggagctggtgctggatacggagcaggagcaggaagcggagctggctcaggtgccggtgctggttcaggagctggagcaggatatggagcaggagctggtgctggatacggagcaggatatggagcaggagctggtgctggatacggagcaggagcaggaaccggagctggctcaggtgctggtgctggttcaggtgctggtgctggttcaggtgctggtgctggttcaggagctggagctggttcaggtgccggtgctggttcaggtgctggtgctggttcaggtgccggttctggttcaggtgctggtgctggttcaggagctggagctggttcaggtgccggtgctggttcaggtgctggtgctggttcaggtgctggtgctggttcaggagctggagctggatacggagcaggagctggtgctggatacggagcaggatatggagcaggagctggtgctggatacggagcaggagcaggaagcggtgctggttcaggtgctggtgctggttcaggtgctggtgctggttcaggtgctggtgctggttcaggagctggtgctggatatggagctggatacggagcaggagctggaagcggagctgcctctggtgctggtgccggtgctggagctggtgcaggaacaggctcttctggatttggaccatatgtagcacatggcggatatagcggctacgaatacgcttggtcgtcagaatctgactttggaactggaagcggagctggtgctggttcaggtgctggtgctggcgcaggtgctggtgctggttcaggagctggagctggatacggagcaggagttggtgctggatacggagcaggatatggagcaggagctggtgctggatacggagcaggagcaggaagcggaactggctcaggtgctggtgctggttcaggagctggagctggatacggagcaggagttggtgctggatacggagcaggagcaggaagcggagctgcctttggtgccggtgctggtgctggtgctggttcaggtgccggtgctggttcaggtgctggtgctggttcaggtgctggtgctggttcaggtgctggtgctggttcaggagctggtgctggatacggagcagggtacggagcaggagttggtgctggatacggagcaggagctggaagcggagctgcctctggtgccggtgctggttcaggtgctggtgctggttcaggtgccggtgctggttcaggtgctggtgctggctcaggtgctggtgctggttcaggagctggagctggatacggagcaggagttggtgctggatacggagcaggatatggagcaggagctggtgctggatacggagctggagcaggaagcggagctgcctctggtgccggtgctggttcaggtgctggtgctggtgctggttcaggtgccggtgctggttcaggtgctggtgctggttcaggtgctggtgctggttcaggtgctggttcaggtgctggtgctggttcaggtgccggtgctggttcaggagctggtgctggatacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggttcaggtgctggtgctggttcaggtgctggtgctggctcaggtgctggtgctggttcaggagctggagctggatacggagcaggagttggtgctggatacggagcaggatatggagcaggagctggtgctggatacggagcaggagcaggaagcggagctggctcaggtgctggtgctggttcaggagctggagctggttcaggtgccggtgctggttcaggtgctggtgctggttcaggtgccggtgctggttcaggtgctggggctggttcaggagctggtgctggatacggagcaggagcaggaagcggagctgcctctggtgccggtgctggttcaggtgctggtgctggttcaggtgccggtgctggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctggctcaggtgctggtgctggttcaggtgctggtgctggttcaggtgccggtgctggttcaggtgctggtgctggttcaggtgccggttcaggtgctggtgctggttcaggagctggtgctggttcaggtgctggtgctggttcaggtgctggttcaggtgctggtgctggctcaggtgctggtgctggatacggagtaggatatggagcaggagctggtgctggatacggagcaggagcaggaagcggagctggctcaggtgctggtgctgggtcaggtgccggtgctggttcaggtgctggtgctggttcaggtgccggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctggctcaggtgctggtgctggttcaggtgctggtgctggttcaggtgccggtgctggttcaggtgctggtgctggttcaggagctggtgctggttcaggtgccggtgctggttcaggtgctggtgctggttcaggtgccggttcaggtgctggtgctggttcaggagctggtgctggttcaggtgccggtgctggttcaggtgctggtgctggttcaggtgccggttcaggtgctggtgctggttcaggagctggtgctggttcaggtgctggtgctggttcaggagctggagctggatacggagcaggagttggtgctggatacggagtaggatatggagcaggagttggtgctggatacggagcaggagcaggaagcggagctgcctctggtgccggtgctggttcaggtgctggtgctggtgctggttcaggtgccggtgctggttcaggtgctggtgctggttcaggtgctggtgctggttcaggtgctggtgctggttcaggtgctggtgctggttcaggagctggtgctggatacggagcagggtacggagcaggagttggtgctggatacggagcaggagctggcgttggatacggagcaggagctggcgctggatacggagcaggagctggaagcggagctgcctctggtgccggtgctggtgctggttcaggtgccggtgctggtacaggtgctggggctggttcaggagctggtgctggatacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggttcaggtgctggtgctggttcaggtgctggtgctggctcaggtgctggtgctggttcaggagctggagctggatacggagcaggagttggtgctggatacggagcaggagcaggaagcggagctggctcaggtgctggtgctggttcaggagctggagctggttcaggtgctggtgctggttcaggagctggtgctggttcaggagctggagctggatacggagcaggagcaggaagcggaactggctcaggtgctggtgctggttcaggtgctggtgctggttcaggtgccggtgctggttcaggtgctggtgctggttcaggagctggtgctggttcaggtgctggtgctggttcaggagttggtgctggatacggagtaggatatggagcaggagctggtgctggatacggagtaggatatggagcaggagctggtgctggatacggagcaggagcaggaagcggaactggctcaggtgctggtgctggttcaggtgctggtgctggttcaggtgccggtgctggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctggctcaggtgctggtgctggttcaggtgctggtgctggttcaggtgccggtgctggttcaggtgctggtgctggttcaggtgccggttcaggtgctggtgctggttcaggagctggtgctggttcaggtgctggtgctggttcaggtgctggttcaggtgctggtgctggctcaggtgctggtgctggatacggagtaggatatggagcaggagctggtgctggatacggagcaggagcaggaagcggagctggctcaggtgctggtgctgggtcaggtgccggtgctggttcaggtgctggtgctggttcaggtgccggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctggctcaggtgctggtgctggttcaggtgctggtgctggttcaggtgccggtgctggttcaggtgctggtgctggttcaggagctggtgctggttcaggtgccggtgctggttcaggtgctggtgctggttcaggtgccggttcaggtgctggtgctggttcaggagctggtgctggttcaggtgctggtgctggttcaggagctggagctggatacggagcaggagttggtgctggatacggagtaggatatggagcaggagctggtgctggatacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggttcaggtgctggtgctggttcaggagctggtgctggatacggagcagggtacggagcaggagttggtgctggatacggagcaggagctggcgttggatacggagcaggagctggcgctggatacggagcaggagctggaagcggagctgcctctggtgccggtgctggtgccggtgctggtgctggttcaggtgccggtgctggttcaggtgctggtgctggtgctggttcaggagctggtgctggatacggagcagggtacggaataggagttggtgctggatacggagcaggagctggcgttggatacggagcaggagctggcgctggatacggagcaggagctggaagcggagctgcctctggtgccggtgctggttcaggtgctggtgctggttcaggtgctggtgctggttcaggtgctggtgctggttcaggtgctggtgctggttcaggtgctggtgctggttcaggagctggtgctggatacggagcagggtacggagcaggagttggtgctggatacggagcaggagctggcgttggatacggagcaggagctggcgctggatacggagcaggagctggaagcggagctgcctctggtgccggtgctggtgccggtgctggtgctggtgctggttcaggtgccggtgctggttcaggtgctggtgctggttcaggtgctggtgctggttcaggagctggtgctggttcaggtgctggtgctggttcaggtgctggtgctggttcaggtgctggtgctggctcaggtgctggtgctggttcaggagctggagctggatacggagcaggagttggtgctggatacggagcaggatatggaggagctggtgctggatacggagcaggagcaggaagcggagctgcctctggtgccggtgctggttcaggtgctggtgctggttcaggagctggtgctggttcaggtgctggtgctggttcaggtgctggggctggttcaggtgctggtgctggatacggagcaggagcaggaagcggagctgcctctggtgctggtgccggtgctggagctggtgcaggaacaggctcttctggatttggaccatatgtaaatggcggatatagcggctacgaatacgcttggtcgtcagaatctgactttggaactggaagcggagctggtgctggctcaggtgctggtgctggttcaggagctggagctggatacggagcaggagttggtgctggatacggagcaggatatggagcaggagctggtgctggatacggagcaggagcaggaagcggagctgcctctggtgccggtgctggttcaggtgctggtgctggttcaggtgccggtgctggttcaggtgctggtgctggttcaggtgctggttcaggtgctggtgctggttcaggtgctggtgctggttcaggtgctggtgctggttcaggtgctggtgctggttcaggtgctggtgctggttcaggagctggagctggatacggagcaggagttggtgctggatacggagcaggatatggagcaggagctggtgctggatacggagcaggagcaggaagcggagctgcytctggcgccggtgctggttcaggtgctggtgctggtgctggttcaggtgccggtgctggttcaggtgctggtgctggttcaggtgctggtgctggttcaggtgctggtgctggttcaggtgctggtgctggttcaggtgctggttcaggtgctggtgctggttcaggagctggtgctggatacggagcagggtacggagcaggagttggtgctggatacggagcaggagctggcgttggatacggagcaggagctggcgctggatacggagcaggagctggaagcggagctgcctctggtgccggtgctggttcaggttctggtgctggttcaggtgccggtgctggttcaggtgctggtgctggttcaggtgctggtgctggtgctggttcaggtgctggggctggttcaggagctggtgctggttcaggagctggtgctggatacggagcagggtacggagcaggagcaggaagcggagctgcctctggtgctggtgccggtgctggagctggtgcaggaacaggctcttctggatttggaccatatgtagcaaatggcggatatagcggctacgaatacgcttggtcgtcagaatctgactttggaactggaagcggagctggtgctggctcaggtgctggtgctggttcaggagctggagctggatacggagcaggagttggtgctggttacggagcaggatatggagcaggagctggtgctggatacggagcaggagcaggaagcggagctggctcaggtgccggtgctggttcaggtgctggtgctggttcaggtgccggtgctggttcaggtgctggtgctggttcaggagctggtgctggttcaggtgctggtgctggttcaggtgctggtgctggttcaggtgctggtgctggatacggagcaggatacggagcaggagctggtgctggatacggagcaggagctggcgttggatacggagcaggagctggcgctggatacggagcaggagctggaagcggagctggctctggtgccggtgctggttcaggttctggtgctggtgctggttcaggttctggtgctggttcaggtgctggtgctggttcaggtgctggtgctggttcaggtgctggtgctggttcaggagctggtgctggttcaggtgctggtgctggttcaggtgctggtgctggttcaggtgctggtgctggatacggagcagggtacggaataggagttggtgctggatacggagcaggagctggcgttggatacggagcaggagctggcgctggatacggagcaggagctggaagcggagctgcctctggtgccggtgctggttcaggtgctggtgctggttcaggagctggtgctggttcaggtgctggtgctggttcaggtgctggtgctggttcaggtgctggtgctggttcaggagctggtgctggttcaggtgctggtgctggttcaggtgctggtgctggttcaggagctggtgctggatacggagcaggagctggcgttggatacggagcaggagctggaagcggagctgcctctggtgctggtgctggttcaggtgctggtgctggttcaggtgctggtgctggttcaggtgctggtgctggttcaggtgctggtgctggttcaggtgctggtgctggttcaggtgctggtgctggttcaggtgctggttcaggtgctggtgctggctcaggtgctggtgctggatacggagcagggtacggagcaggagttggtgctggatacggagcaggtgctggatacggagcaggatatggagtaggagctggtgctggatacggagcaggagcaggaagcggagctggctctggtgctggtgctggttcaggtgctggtgctggttcaggtgctggtgctggttcaggtgctggtgctggttcaggtgctggtgctggttcaggtgctggttcaggagctggtgctggatacggagcaggagctggcgctggatacggagcaggagctggcgctggatacggagcaggagctggaagcggagctgcctctggtgctggtgctggtgccggtgctggttcaggtgctggtgctggttcaggtgctggtgctggttcaggtgctggttcaggtgctggtgctggttcaggagctggtgctggatacggagcaggagctggaagcggagctgcctctggtgccggtgctggttcaggtgctggtgctggtgctggtgccggtgctggtgctggttcaggagctggtgctggttcaggagctggtgctggatacggagcaggagcaggaagtggagctgcctctggtgctggtgctggagctggtgcaggaacaggctcttctggatttggaccatatgtagcaaatggcggatatagcagacgtgaaggctacgaatacgcttggtcgtcaaaatctgactttgaaactggaagcggtgctgcctctggtgctggtgctggtgctggttcaggtgctggtgctggttcaggtgccggtgctggttcaggtgctggtgctggttcaggtgccggtgctggtggtagcgtcagttacggagctggcaggggatacggacaaggtgcaggaagtgcagcttcctctgtgtcatctgcttcatctcgcagttacgactattctcgtcgtaacgtccgcaaaaactgtggaattcctagaagacaactagttgttaaattcagagcactgccttgtgtgaattgctaatttttaatataaaataacccttgtttcttacttcgtcctggatacatctatgttttttttttcgttaataaatgagagcatttaagttattgtttttaattacttttttttagaaaacagatttcggattttttgtatgcattttatttgaatgtactaatataatcaattaatcaatgaattcatttatttaagggataacaataatccatgaattcacatgcacatttaaaacaaaactaaattacaataggttcatataaaaacaacaagtatgccttctcaactaagaatactatattgtttaaaccgtaaaaaaagtcatatttctgtatatcaaaacacatctaatattaaaaaaacagtcagcaagcacttacaagtgtgggctcggacagcaattacctggtctcaggagacacttgaagaacgagaagcacgtctctctgtcgattgcgaggctcatgcactatcgcttgagtctgagacctttactgatagggaaatccgtttgagctctcagagggttcggacagcaaaagcttgcccggtctcaggagacattagaagaacgggaagcataactcaataccgatcgcgtttccattgagcctatgcttcgtgataataataaataaagcccaaggtcagacgctaaaagttgtgggtgtagatataagtgttaactgcttttcacacagtcaactcggtcacgcggttatgtagctctgtcacatgctagcaatcctagcaacctgtatgctttgatccccgatggctatacgtctacgtcgtgtatagagaagctt

SEQ ID NO: 2 - DNA, 25 bp, Bombyx mori
  misc_feature (4)..(4): n is a, c, g, or t
yttntggagctggtgcaggtgcagg

SEQ ID NO: 3 - DNA, 25 bp, Bombyx mori
  misc_feature (4)..(4): n is a, c, g, or t
tyyncagttccaaagtcagattctg

SEQ ID NO: 4 - DNA, 25 bp, Bombyx mori
  misc_feature (4)..(4): n is a, c, g, or t
yttngaccatatgtagcacatggcg

SEQ ID NO: 5 - DNA, 25 bp, Bombyx mori
  misc_feature (4)..(4): n is a, c, g, or t
yttncagctcctgctccgtatccag

SEQ ID NO: 6 - DNA, 23 bp, Bombyx mori
  misc_feature (21)..(21): n is a, c, g, or t
gctggtgctggatacggagcngg

SEQ ID NO: 7 - DNA, 23 bp, Bombyx mori
  misc_feature (21)..(21): n is a, c, g, or t
gctggaagcggagctgcctcngg

SEQ ID NO: 8 - DNA, 23 bp, Bombyx mori
  misc_feature (21)..(21): n is a, c, g, or t
accagcaccagcacctgaacngg

SEQ ID NO: 9 - DNA, 7308 bp, Unknown
  repeating motifs of MaSp2 and flagelliform
cccccaggcggtgcaggtccaggcggtgcgggtccaggcggagcaggcccaggcggagcaggtccaggcggagcgggtccaggtggtgctggccccggtggagccggtcctggcggtgctggacctggaggcccatcaggtcctggttctgctgccgcagctgctgccgctgccggcccaggtggagccggtccgggcggtgctggacccggaggcgccggccctggtggagctggtccaggcggtgccggaccgggaggcgctggccccggtggagccggtcctggcggtgctggacccggaggccctagcggaccaggctccgctgctgcagccgcagctgcagctggcccaggtggagccggtccgggcggtgctggacccggaggcgccggccctggtggagctggtccaggcggtgccggaccgggaggcgctggccccggtggagccggtcctggcggtgctggaccgggaggcccctcgggtcctggaagtgctgccgcagcagcagctgcagcaggcccaggtggagccggtccgggcggtgctggacccggaggcgccggccctggtggagctggtccaggcggtgccggaccgggaggcgctggccccggtggagccggtcctggcggtgctggtcctggtggtccttcaggccccggttctgctgccgcagcagccgctgcagcgggcccaggtggagccggtccgggcggtgctggacccggaggcgccggccctggtggagctggtccaggcggtgccggaccgggaggcgctggccccggtggagccggtcctggcggtgctggacctggaggccccagcggaccgggctctgcagctgccgctgctgctgctgctggcccaggtggagccggtccgggcggtgctggacccggaggcgccggccctggtggagctggtccaggcggtgccggaccgggaggcgctggccccggtggagccggtcctggcggtgctggacccggaggcccctcgggtccaggaagtgctgccgcagcggcagctgcagccggcccaggtggagccggtccgggcggtgctggacccggaggcgccggccctggtggagctggtccaggcggtgccggaccgggaggcgctggccccggtggagccggtcctggcggtgctggaccgggaggcccttcaggccctggttctgctgccgccgctgcagctgccgcaggcccaggtggagccggtccgggcggtgctggacccggaggcgccggccctggtggagctggtccaggcggtgccggaccgggaggcgctggccccggtggagccggtcctggcggtgctggtcctggtggtccgagcggacccggctctgcagctgcggctgccgctgcagctggcccaggtggagccggtccgggcggtgctggacccggaggcgccggccctggtggagctggtccaggcggtgccggaccgggaggcgctggccccggtggagccggtcctggcggtgctggacctggaggcccttctggtccgggaagtgctgccgcagccgcagctgccgccggcccaggtggagccggtccgggcggtgctggacccggaggcgccggccctggtggagctggtccaggcggtgccggaccgggaggcgctggccccggtggagccggtcctggcggtgctggacccggaggcccatcaggcccaggttctgctgccgcagcggccgctgccgccggcccaggtggagccggtccgggcggtgctggacccggaggcgccggccctggtggagctggtccaggcggtgccggaccgggaggcgctggccccggtggagccggtcctggcggtgctggaccgggaggcccaagcggacctggctccgctgctgccgccgcagctgcggctggcccaggtggagccggtccgggcggtgctggacccggaggcgccggccctggtggagctggtccaggcggtgccggaccgggaggcgctggccccggtggagccggtcctggcggtgctggtcctggtggtccttctggtcccggaagtgctgccgccgctgccgctgcggcaggcccaggtggagccggtccgggcggtgctggacccggaggcgccggccctggtggagctggtccaggcggtgccggaccgggaggcgctggccccggtggagccggtcctggcggtgctggacctggaggcccgtctggtcctggttcagctgccgcagcagcagcagcggcgggcccaggtggagccggtccgggcggtgctggacccggaggcgccggccctggtggagctggtccaggcggtgccggaccgggaggcgctggccccggtggagccggtcctggcggtgctggacccggaggcccgtctggaccaggctctgctgccgcagcagcggctgcggccggcccaggtggagccggtccgggcggtgctggacccggaggcgccggccctggtggagctggtccaggcggtgccggaccgggaggcgctggccccggtggagccggtcctggcggtgctggaccgggaggcccgtcaggtcctggatctgctgccgcagcagctgctgcagccggcccaggtggagccggtccgggcggtgctggacccggaggcgccggccctggtggagctggtccaggcggtgccggaccgggaggcgctggccccggtggagccggtcctggcggtgctggtcctggtggtcctagcggccccggttcagcagccgctgccgcagcagctgcgggcccaggtggagccggtccgggcggtgctggacccggaggcgccggccctggtggagctggtccaggcggtgccggaccgggaggcgctggccccggtggagccggtcctggcggtgctggacctggaggcccctctggaccgggctctgcggctgcagctgctgctgcagctggcccaggtggagccggtccgggcggtgctggacccggaggcgccggccctggtggagctggtccaggcggtgccggaccgggaggcgctggccccggtggagccggtcctggcggtgctggacccggaggccccagcggtccaggatctgcagccgctgccgcagcggcggcgggcccaggtggagccggtccgggcggtgctggacccggaggcgccggccctggtggagctggtccaggcggtgccggaccgggaggcgctggccccggtggagccggtcctggcggtgctggaccgggaggccccagcggccctggttcagcagctgcagccgcagctgctgcaggcccaggtggagccggtccgggcggtgctggacccggaggcgccggccctggtggagctggtccaggcggtgccggaccgggaggcgctggccccggtggagccggtcctggcggtgctggtcctggtggtccaagcggacccggctctgcggctgccgctgccgctgcagctggcccaggtggagccggtccgggcggtgctggacccggaggcgccggccctggtggagctggtccaggcggtgccggaccgggaggcgctggccccggtggagccggtcctggcggtgctggacctggaggcccctctggtccgggatctgcagctgcagccgccgctgctgccggcccaggtggagccggtccgggcggtgctggacccggaggcgccggccctggtggagctggtccaggcggtgccggaccgggaggcgctggccccggtggagccggtcctggcggtgctggacccggaggccccagtggcccaggttcagcagctgccgctgcagctgctgccggcccaggtggagccggtccgggcggtgctggacccggaggcgccggccctggtggagctggtccaggcggtgccggaccgggaggcgctggccccggtggagccggtcctggcggtgctggaccgggaggccccagtggacctggctccgctgccgcagccgcagcagcagccggcccaggtggagccggtccgggcggtgctggacccggaggcgccggccctggtggagctggtccaggcggtgccggaccgggaggcgctggccccggtggagccggtcctggcggtgctggtcctggtggtccttctggtcccggatctgctgccgcagcggcggctgctgcgggcccaggtggagccggtccgggcggtgctggacccggaggcgccggccctggtggagctggtccaggcggtgccggaccgggaggcgctggccccggtggagccggtcctggcggtgctggacctggaggccccagtggtcctggttccgcagccgctgccgcagccgccgccggcccaggtggagccggtccgggcggtgctggacccggaggcgccggccctggtggagctggtccaggcggtgccggaccgggaggcgctggccccggtggagccggtcctggcggtgctggacccggaggccccagcggaccaggctctgcagccgctgccgccgctgctgccggcccaggtggagccggtccgggcggtgctggacccggaggcgccggccctggtggagctggtccaggcggtgccggaccgggaggcgctggccccggtggagccggtcctggcggtgctggaccgggaggccccagcggtcctggatcagcagccgctgccgccgcggcagcgggcccaggtggagccggtccgggcggtgctggacccggaggcgccggccctggtggagctggtccaggcggtgccggaccgggaggcgctggccccggtggagccggtcctggcggtgctggtcctggtggtcctagtggccccggttcagccgctgcagccgcagcagccgcgggcccaggtggagccggtccgggcggtgctggacccggaggcgccggccctggtggagctggtccaggcggtgccggaccgggaggcgctggccccggtggagccggtcctggcggtgctggacctggaggccccagcggaccgggctctgctgctgccgctgcagctgctgctggcccaggtggagccggtccgggcggtgctggacccggaggcgccggccctggtggagctggtccaggcggtgccggaccgggaggcgctggccccggtggagccggtcctggcggtgctggacccggaggccccagcggtccaggatctgctgctgcagctgcagctgcagctggcccaggtggagccggaccgggcggtgctggtcccggaggcgccggacctggtggagctggtccaggcggtgccggaccgggaggcgctggtcccggtggagccggacctggcggtgctggtccgggaggcccctctggccctggttcagctgccgcggctgctgccgctgccggaccaggtggagccggtccgggcggtgctggacccggaggcgccggtcctggtggagctggaccaggcggtgccggtccgggaggcgctggacccggtggagccggtcctggcggtgctggtcctggtggtccttctggacccggctctgctgctgctgctgctgccgctgccggtccaggtggagctggaccgggcggtgctggtcccggaggcgctggacctggtggagctggtccaggcggtgctggaccgggaggcgctggtcccggtggagctggacctggcggtgctggtcctggaggcccatctggtccgggatctgcggctgcagctgctgctgccgctggaccaggtggagctggtccgggcggtgctggacccggaggcgctggtcctggtggagctggaccaggcggtgctggtccgggaggcgctggacccggtggagctggtcctggcggtgctggacccggaggccccagcggcccaggttcagcggctgccgctgcggctgcagctggtccaggtggagctggaccgggcggtgctggtcccggaggcgctggacctggtggagctggtccaggcggtgctggaccgggaggcgctggtcccggtggagctggacctggcggtgctggtccgggaggccctagcggacctggctctgcagctgcagctgccgctgccgcaggaccaggtggagctggtccgggcggtgctggacccggaggcgctggtcctggtggagctggaccaggcggtgctggtccgggaggcgctggacccggtggagctggtcctggcggtgctggtcctggtggtccttctggtcccggatctgcagctgcagctgcggctgctgctggtccaggtggagctggaccgggcggtgctggtcccggaggcgctggacctggtggagctggtccaggcggtgctggaccgggaggcgctggtcctggtggagctggaccaggcggtgctggtcctggaggccctagtggtcctggttctgcagctgcagctgctgctgctgctggaccgggtggagctggtcctggcggtgctggtcctggtggtgctggtccgggtggagctggacctggcggtgctggtcctggtggtgctggaccgggtggagctggtcctggcggtgctggacccggaggccccagcggaccaggctctgcggctgcggctgctgctgccgcaggtccaggtggagctggaccgggcggtgctggtcctggaggcgctggaccaggtggagctggtccgggcggtgctggacctggaggcgctggtccaggtggagctggaccgggcggtgctggtccgggaggcccatctggtcctggatctgcagctgcggctgctgctgctgctggacctggtggagctggtccaggcggtgctggaccgggaggcgctggtcctggtggagctggaccaggcggtgctggtccgggaggcgctggacctggtggagctggtccaggcggtgctggtcctggtggtcctagcggccccggtagcgctgctgccgctgccgctgccgctggtccgggtggagctggacctggcggtgctggtcctggtggtgctggaccgggtggagctggtccaggcggtgctggacctggaggcgctggtccaggtggagctggacctggcggtgctggtcctggaggcccgtctggaccgggctcagcagctgcagctgccgcagcagccggaccaggtggagctggtcctggcggtgctggtcctggtggtgctggtcctggtggagctggtccaggcggtgctggtcctggaggcgctggtcctggtggagctggtcctggaggagcaggtccaggtggtccctcaggtccaggttcggcggcggcggcggcagcagcagcg

SEQ ID NO: 10 - DNA, 3156 bp, Caerostris darwini
gccgggggtgcgggcggacgaggaggattaggggggcagggcgggggtcagggcgctggaggagccggacagggaggctatggaagcggcttgggaggcttagggggcggagccgctgcagctgcagccgctgccggcggtgcaggcggcctgggcgggcaagggggtggccaaggcgctggccaaggcggctacggatcaggtcagggtggccaaggcgccggaagcgcggctgccgcggccgctgcggggggcgcaggcggtaggggggggctagggggtcagggcggtgggcagggtgccggtggcgccggtcaggggggatacggatctggactggggggcttgggcgggggggcagcggcggccgcggccgcagccggaggagcaggagggctcggtggacaaggcggagggcagggcgctggtcaaggcggctatggttccggccaaggagggcaaggagccggctcggctgccgcagcagcggccgcgggtggtcgaggaggatatggtgggcaagggggcgggcaaggagcggggggtgctgggcaaggtggttacgggagtgggcttggtggactgggtggtggcgcagcagccgcggcggcggcggcgggaggcgctggcggcctcggaggacagggaggaggtcaaggcgctgggcagggaggctacggtagcggtcagggcggtcagggcgccgggtccgctgcggccgcggccgctgccggaggagccggcggccttggcgggtatgggggccaggggggaggacagggggcgggaggagctggccaggggggttatgggtcaggtttagggggccttggcggtggagcagccgcggccgctgcagcagcgggtggcgcgggtgggctggtcggtcagggcggtgggcagggcgcagggcagggagggtatggtagtgggcaaggagggcaaggcgctgggtcagcagcggctgcagccgcagcgggaggtgctgggggcagaggcggtcttggtgggcagggcgggggccagggcgccggcggggcagggcaagggggttatggaagcgggctcgggggtttgggtgggggcgcgtcagctgcggcagctgctgcggggggtgccggagggctcggaggacaggggggtggccaaggtgcgggccaggggggatacggaagcggccaaggggggcagggagctgggtctgccgcagctgctgcggcagcggggggatcaggcggattggggggacagggcggatacggtgggcaggggggctacggcggtggctatggcgggcaacagcaggggggggggcagggggctgggcaagggggttatggatcaggccagggaggtcagggcgcgggttccgcggcagctgctgcagccgcggggggacgtggaggatacggcgggcagggcggaggacagggtgcgggtggggccggacaaggcggatatggatctggcttgggtggactagggggcggagccgcggcagctgcggggggagcgggaggactcggaggacagggagggggccaaggggctggacaaggaggctacggatcggggcagggcgggcagggcgctggatcagctgcggcggccgctgctgctgggggaagaggcggttatggtgggcaggggggcgggcagggcgctgggggcgctggccaagggggctatggctccggtctggggggcctcggaggtggagctgccgctgctgcagcagcggccggcggtgcgggtggtctcgcagggcaaggtggtggacagggagccggccaaggagggtatggatcaggtcagggaggccagggtgctggctctgcagcagcggctgccgcggccggaggcgccgggggacgcggtggctatgggggtcaagggggacagggtgctggaggtgctggccaaggcggatatggctctggccttggcggtctaggggggggggctgccgccgcggcagcagcggcggggggtgcagggggcctgggcgggcagggtgggggccagggtgctggtcagggtggctatggttcgggtcaggggggacaaggcgcaggctccgcagcggcagcggccgctgcaggaggcgcggggggacaggggggatacggtggacaagggggccagggggcgggtggggctggtcaaggcggttacggttcgggtcttggaggtgtcgggggtggcgctgcggctggaggcgcaggcggattgggaggtcaggggggcggtcagggcgcaggtcagggtgggtatggctcagggcagggaggccagggcgcaggcagtgcggcggcagcgggacgcggcgggtacggcgggcgtgggggcgcaggcagctcttcggcagcagctgctgcaggttcggggggtgatggtagcggcagtggggggtacggtgggcgcgggggtcaaggaggtgatggcgcgtcttcggctgccgcagcggctggaagcgggggtgatggatccggatcaggaggttacggcgggaggggcggacgaggcgggcagggtggggcggggagcagtagtgccgccgcggcggcagggagcggaggagatggatcgggaagcgggggttacggagggcgaggaggtaggggcggggctggatcttcgagtgccgcgagcgcagctgcgggaggtgaggatggattcggtcgtggcgggtacggtggtagggggggagcagggtcctcttcggccgctgcagccgcaggcagcggcggagacgggtccggttcgggggggtatggcgggagaggtggtcagggcggagcgggtagtagttcagcagctgctgctggttctgggggcgatgggtacgggtctggcggatatggtgggcgaggagggcaaggaggggcaggatcttctagtgcagccagcgcagctggaagtggaggtgatgggttcggatcgggtttttacgggggtagagggggggaaggtggcgccggaagctcttccgcggcggcagcagctggcagtggaggggacggctacgggtccgggggatacggcggacgcggaggacaggggggtgcgggaggcgcttcggcttcagccgtcgcagcgggaggcggccgtgggcagggggggtatggcgggaggggtggtcaagggggagcagga

SEQ ID NO: 11 - DNA, 8484 bp, Latrodectus hesperus
ggacaagggggcgcgggacctggtggtgcaggtgccgcggcagcagcggcggccgcggctggcggggccgggcaaggcggtcaagggggatacggccaaggtggttacggtcaagggggggcgggtcaaggcggcagcggtgccgcggcagcggcagccgcagcagccggcgggactggacaaggcggagcgggtcagggtggggccggagcagcagcagctgctgccgcggcggccggcggagcgggccaaggagggcaaggcggctatggccaaggcggctatggacagggaggaacgggccaaggtggagcgggtgctgctgcagctgccgcggcagcggggggagcgggacaaggaggccaaggtggctatggccaggggggatatggccagggaggatacggtcagggtggttccggcgcggctgccgccgcggcagcggcggccggaggcgcgggtcaaggtggtcaaggcggatacggacaggggggctatggccaagggggcgcagggcaagggggcgcaggagcggccgctgctgctgcggcggctgcgggcggtgctgggcagggaggatatggacgcggtggtgcaggccaaggaggtgctgcggcagccgctgccgcagcggccggtgcaggacaaggagggtatggcggtcaaggtgcggggcaaggaggaagcggagccgccgctgcagcggcagcggcaggtggcgcaggccagggaggtcaaggtggctatggacagggaggttacggccagggaggttcaggcgctgctgcagctgctgcagccgccgggggcgctggccagggaggtcaaggcggctatggccaagggggctacggccaaggtggggctggtcaagggggcgcaggagccgctgcagctgcagcggcggcggggggagctgggcaggggggtcaaggcggttatggccaaggaggatacggtcaaggaggagcgggacaaggtggtgcgggggccgcggccgccgcagccgctgcggggggagccggtcaaggtgggcagggcgggtatggtcagggcggttatggtcaaggaggggccggtcagggcggtgctggcgctgccgcggctgctgctgcagccgggggggccggacaaggggggcagggaggttatgggcaggggggatacggtcagggtggagcgggacagggtggtgccgcggccgcggcggcggcggccgctggtggtgcgggtcaagggggctatggccgtggaggtgcgggacagggcggggccgctgctgcagcaggtgctggacagggtggatatggcggacagggcgcaggccaaggaggtgcaggagcagctgcggctgcggcggccgccggcggtgcgggacagggtggccaggggggctatggaaggggtgggtacggtcaaggaggagcgggacaaggcggggcgggcgcagcagcggccgccgccgcggcgggcggcgcggggcagggtgggcaaggcggctacggccaaggagggtacggacagggtggcgctggccaaggcggtgccgcggctgctgccgctgcggcgggaggtgcaggtcagggcggatacggtagggggggcgctggtcaagggggggctgcggccgccgcggctgctgccgcgggtgctggccaaggaggctacggcggtcaaggagcgggccagggcggagctggtgccgctgcggccgcggccgctgctggaggcgcaggccaaggagggcagggagactacggcagaggaggatatgggcaaggaggggccgggcaggggggcgcgggcgccgcggcggccgccgctgctgctggcggtgcaggtcagggtgggcagggaggttatgggcagggcggatacggccagggtggggccgggcaggggggtgcagcagcggcagcttctgccgctgctgcaggcggagctggacagggaggatacgggagaggtggcgcaggccaaggcggagctgccgcggcggccggggcgggacaaggggggtacgggggtcagggcgctggccaaggcggcgccggagctgccgcggcagctgcggctgccggaggagcgggacagggaggccaaggcggttacggacgtggaggttatggtcaagggggagcgggacaggggggggcgggcgcagccgctgcagccactgcagcagggggtgcgggtcaaggaggacagggaggatatggtcagggtggttatggtcaaggcggagctggccagggcggggcggctgctgccgcagcggctgcagcgggaggcgccgggcaggggggctatggccgtgggggggctggacaaggaggcgccgcagccgcagccgccgctgccgcaggagccggtcagggcggctacggcgggcaaggagctggacaggggggagcgggagcagctgcagcagcggctgggggagccggacaaggtggacaaggcggttatggaaggggggggtacggacaaggcggcgcgggccaggggggcgctggagcggcagctgcggcagccgcggcgggtggagcgggtcagggtggacaaggtggttacggccaaggcggatatggtcaaggaggtgccggacaggggggtgcggcagccgcagccgcggcggcagcgggcggggcaggtcagggcggctacgggaggggtggcgctggtcaaggaggcgcagctgcggccgcaggcgctgggcagggcgggtatgggggacagggtgccgggcaaggtggtgctggcgccgcggcagcggcatcgaggggagctggtcagggagggcagggagggtacggacgcggtggttatggccagggaggcgccgggcaagggggagcaggagccgccgcagctgcggccgccgctggtggcgccggtcaggggggacagggtggttatgggcaaggcgggtacgggcaaggtggtgcgggtcaagggggtgctgcagccgcggcggccgctgcggggggggcgggacagggcggctacggtcgcggcggggcaggacagggcggtgccgcagccgctgccggtgccggccaagggggttatgggggtcagggcgcgggtcaggggggtgctggggccgctgcagcggcagcggccgcagggggcgcaggccagggggggcaggggggctatggcaggggcggatacggtcagggaggggctgggcagggtggagcaggggcggcagccgcagcagcagctgccggtggtgcagggcaggggggccaaggtggatacggtcagggcggctatgggcagggcggcgctgggcaaggaggtgcggcggcggccgctgcggctgcagctggtggtgccgggcaggggggctatggcagggggggcgcgggacaaggcggggctgctgcagctgccgcagccgcggcagggagcggccaaggcggttacggcggccagggagcgggtcaggggggagctggcgccgccgctgcagcggcggctgcgggtggtgctggccaaggtggtcagggtgggtacggccgcggcggatacggccagggtggggcgggccaggggggagcgggtgccgcagccgcggcagcggcagctggaggcgcgggtcaaggcggacagggtggttatggacagggtgggtatggacagggtggggcaggacaaggaggagcggcagctgcggctgcagcagcagcagcgggcggggccgggcaaggcgggtatgggaggggcggagccgggcaaggcggggcagcagcagcggccggagcgggacaaggtgggtatggcgggcagggggctggtcaagggggggctggagcagcggccgcagcggcagccgctggaggcgcaggtcaaggtgggcaaggaggatacggtcgtggtggctacgggcagggaggtgcgggtcagggtggcgctggtacagcggcggctgcggcagcggccggaggggcgggccagggagggcagggcggatatgggcaaggtggttatgggcaaggaggagccggtcaaggaggggctgcggctgcagccgcagcagccgcgggaggagctggtcaaggcgggtacgggagggggggagcagggcaggggggtgcagccgcagcggctgccgccgcggcgggcgcaggacaaggcggatatggagggcagggggcgggccagggcggtgctggcgcagccgccgcggccgcagctgctggtggggcgggtcagggcggtcagggtggttatggcagaggcgggtacggtcagggtggagcgggccaagggggtgcaggggccgcagcagctgcggctgcagctggcggagcgtctcaaggcggtcaaggaggatacggccaaggcgactatggccaagggggtgccgggcaaggtggtgcggcggctgcagctgctgcagcaggaggtgcgggtcagggtggctatggaaggggcggcgctgggcagggaggggcagccgccgccgcaggggccgggcaaggagggtatgggggtcaaggggctggccagggaggcgcgggagctgccgctgctgcagccgctgctggtggcgcaggaaggggaggtcagggaggatacggcaggggggggtacggccaaggcggggctggtcaaggtggcgcgggagctgccgcggcggccgcggccgcgggaggtgcgggccagggtgggcaagggggatacggccaaggagggtacggacagggtgggacgggacaggggggcgcggctgccgcagcagcggcagccgctggcggtgcgggccaagggggttatggaaggggaggtgctggccagggaggcgcagcggccgcggcggcagctgcggcaggtgcaggccaaggaggatacggtggccagggcgcaggccaaggcggggctggggccgcagcggccgcagctgctgctgggggtgctgggcagggggggcaagggggctacggtcgcggtgggtacgggcaaggcggtgctggacaggggggcgcgggcgccgcggcagcggccgctgctgcaggtggggcgggtcagggcggtcagggtggctatggacaaggcgggtacggccagggtgggtatggacaaggtggcgctggccagggtggcgcggctgctgcagcagctgctgcggggggagctgggcagggagggtacggaaggggtggagccgggcaaggtggtgcggcagcggccgcgggtgccggacaaggcgggtacggggggcagggagcgggccaggggggcgcgggggccgccgcagcggcagcagccgcgggtggtgcagggcaaggtggacaaggagggtacggtagggggggttatgggcaaggtggcgctggacaaggaggggcgggcgctgctgccgcggcggctgcagcgggtggtgcgggtcaaggaggccaagggggttatggtcaaggtgggtatggtcaagggggcgcaggccaaggtggagcagctgcggctgcggcagcggcggctgggggagctggacaaggtggctacggaagaggtggggccggtcaaggaggggccgccgctgctgcagctgcagcagcaggtagtggacaaggcgggtacggtggtcaaggcgcaggacagggtggagctggggctgcagctgctgcagcagctgctggcggggcgggacaaggaggccaaggtggctatggacgtggtggctacgggcaggggggtgctgggcaggggggagctggcgctgccgcggccgcagcggcagctggtggtgcgggtcaaggtgggcagggaggctatggccaggggggttacggccagggtggttatggtcaaggtggtgcgggccaaggtggagctgccgcggcggcagcggctgcggcagctggtggagccggtcagggcggctacggacgtggcggggcgggccaaggtggtgcggcggccgccgcgggagctggacaaggcgggtacggtggacagggtgctgggcaggggggggccggcgctgcagcggcagcagctgcagccggcggagcgggtcagggtggccaagggggttatggtcgagggggctacggtcaggggggggcggggcaaggtggagctggagcggcggcagccgctgcggccgccgggggtgctgggcagggaggacagggcggttacgggcaagggggcaatgggcaagggggcgcaggtcaggggggcgcggcagccgccgcagcagccgccggcggggcggggcaggggggatacggaagggggggtgccggtcagggaggtgcagcagcggcggcagcagcagccgctggtgcagggcaagggggctatggaggccaaggcgcagggcagggaggtgccggcgcggcggctgcggcggctgctgctgggggagcagggcaaggaggccagggcggctacgggaggggaggttacggacagggcggagcgggtcaggggggtgctggcgccgctgctgcggcggctgcggcgggcggggcaagtcaaggtggacagggcgggtacgggcaaggggactacgggcaggggggagcagggcaggggggtgcagccgcagcagcggcagcggcgggcggcgccggccagggaggatatggtcgagggggcgctggacaggggggcgctgctgccgcagctggcgctgggcagggcgggtatggagggcaaggcgcggggcaaggcggggcgggagccgcagcagcggcagctgcggccggcggcgcgggccgtggcggtcaagggggatatggtaggggcgggtatggtcagggtggtgccggacaagggggtgcgggggcggcagcggccgcagcggctgctgggggcgctggccaggggggacaaggaggttatggccaggggggctatgggcaaggcggggccggacaaggcggcgcggccgcggctgcggctgctgcagcagggggagctggtcaaggaggttacgggaggggaggtgctggtcagggtggagcagccgctgcggcaggcgcaggtcaaggaggatacggaggtcagggtgctgggcaagggggggcgggagcggctgccgccgctgccgcagccggtggtgcaggacgaggaggccaagggggatacggtagaggcgggtacggccagggtggggctggccaaggcggggcgggagcagctgctgccgcagccgctgccggtggggcaggccagggcggccagggaggctatggacaaggaggttatggtcaagggggagccgggcagggcggggctgctgctgcagctgccgcggcggtggggggagcgggtcaaggaggatacgggcgaggtggcgctggacaaggaggtgccgcagcggcggccgctgctgcagcggccggcagtggacaagggggatacggcggccaaggtgctggccagggaggggcgggagctgcggcagctgccgcggctgccggtggtgccggacagggaggacaggggggttatggcggaggagggtatggccaaggtggagctgggcagggaggagcaggagcggcagcagcagccgccgcagccgggggtgctggacagggcggccaaggtggttacggtcagggtgggtacggtcagggcggagctggccaaggtggtgcggcagctgccgcagctgccgcggctggtggcgccgggcagggcggatatggaagaggtggagcagggcagggtggagcggcagccgctacaggggctggacaggggggctatggcggtcagggagcggggcaaggtggagctggggcggctgccgcggctgcagccgcgggaggtgctggacagggcgggcaaggcggatacgggagggggggttatggtcaaggtggcgcgggacaaggtggggcaggagctgcggctgctgcggctgctgctggcggagcaggtcagggtgggcagggggggtacggtcaaggtggctatgggcaaggcggggcgggtcagggcggtgctgcggccgcagccgcagcggcaggcggagctggacagggaggctacgggcgcggaggcgccggtcagggtggtgctgccgcagccgccgcagcagctgctggcgccggccagggaggttacggcggccagggcgccgggcaagggggcgcgggggcagcggcagcggcggctgccgctggtggtgccggacagggcggtcagggcggatatggtcgaggtggatatggccaaggcggggctggacagggcggagcaggggccgccgcagccggaggagccggtcaaggtggtcaggggggttatggccagggtgggtacggacaagggggagcgggccaggggggggcagcagcggccgcggcggccgcggcggccgggggagctggacagggaggttatggcggttatgggcagcagggcggagctggcgccgccgcagcagccgcttccggaccaggccaaatctattatggcccccagagcgttgcggctcccgcagcggctgcagcc

SEQ ID NO: 12 - DNA, 1293 bp, Nephila clavipes
ctaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggatccgggctaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatggggga

SEQ ID NO: 13 - DNA, 2886 bp, Caerostris darwini
ccccaatctccatatgggccaggtccccaagggcctgggccccaaggcccagggcctcaggggccgtccggacccggcccgcagcgacctcaaggaccggggccacaagggccctatgggcctggaggtgtctctgtagtgtccgcgacggtgagcgggccagggcctcaaggcccgtccggacccggaccccaaggaccgtatggacccggcccacaaggaccgggaccacagggacccggacctcagctgcccggcccacaaggtccttcaggaccaggtccacaaggtccatatggcccaggacctcaaggaccgggaccgcagggacccggacctcaggggccatctggacccggccctcaacagcctcaaggtcccgggcctcaacgcccttacgggccggggggtgtcagcgttgtgagtacaaccgttagtggacccggaccccaagggccactaggacccggcgcgcaggttccctacggacccggtccgcaagttcccgggccacaaggcccgggaccacaaggacccagcgggcccggccctcagcgtccccaaggaccgggacctcagggaccatatggaccaggaggcgtcagtgtagttagccagacagtcagcggtccgggtcctcaaggacccagcggacctggcccccaaggtccatatggtccaggacctcaggggcctgggcctcaggggcctgccccacagggtcctagcggtcctggtccccagcgaccgcaagggccaggcccgcagcgaccgtatggaccagggggaatatcagttgtttctactaccgtatcaggtcctggaccccaggggccttccgccccgggaccgcaagggccctacggacctggtccacaggtaccaggaccgcaggggccggggccccagggacccagtgggcctggtccccaacgaccacaaggtcccggcccccagggaccgtatggccctggaggagtctctgtcgtgtcacagacagtttccggacctgggccccaaggtccgtctggccctggtcctcaagggccgtatggccccggacctcagggaccagggccacaagggccgggtccgcaagggcccagtggggcgggccctcaacgcccccagggtcctggaccacaaggaccttacggccccggcggagtttctgtcgtgtcagcgactgtgtctgggcccggtcctcaagggccgtctggcccgggtcctcaaggtccatatggtcctggaccgcagggtcctggaccgcaaaggccagtcccccaagggcccagtggtcctcgccctcagcaaccacaagggccaggaccacaacgacctttcggtccggggggcgtcagtgcagtaagcacgacagtttttgggcctggacctcaaggtccatccggtcctggtcctcaaggtccctatggtcctgggcctcaaggcccgggtccacaaggtccaggaccgcaaggaccctcgggtcaaggtcctcaacgaccctctggtccgaggccacaagggccctatggacccggtggaatttctgttgtgtcggcgacggtgagtggccctgggccacaagggccgtcgggtcccggtccacagaggccttacggtcctggaccagaaggtccaggtccgcaaggggcagggccacaaggtcccggtttacaaagacccagtgggcctggtccacagggtccttatggcccagggccgcgaggaccgccgcaagggccgtatggtcctggtccccagggacctggcccgcaaggcccaggacctcaaggaccagggccacgaggcccatccggacctgggccgcagggtccgtacggcccaggtggcgtatcagtggtctccgcttccgtgagcggcccaggtccgcaaggacctagcggacccggaccgcagggcccgtatgggccaggacctcagggtcccggaccacagggtcctggtccccagggtccagggccccagggccccggcccccgtggcccaagcggccccggaccgcagggtccgtatggtcccggcggcgtcagtgtggcctcagctagcgttagtggacctgggccccaaggtcccagtggcccgggaccgcaaggcccgtacggcccgggtccacaaggtccgggtcctagagggccgggtccgcagggtcccggcccacagggaccgggacctcagggcccatccggacccggtccacagggtccgtacggacccgggggagtcagcgtcgtatcagccagtgtatctggtccggggccacaggggccctctggtcccggaccgcaagggccatacggccccgggccccaaggccctggccctcaggggcccggcccccaagggccaggacctcaaggtcccgggccaaggggaccatctggacccggcccccaagggccgtacgggcctgggggcgtctctgttgtaagtgctagcgttagcggccctggcccgcaagggccgtctggcccagggcctcagggaccatacggtccggggcctcagggtccaggcccgcaagtgccgggtccacagggccctggcccccagggtccaggtccccaaggaccttcgggtccagggccccaaggcccctatgggcctggcggcgtctctgtagtaagtgcgagtgtttcgggtcctggaccacagggtcccagcgggcccggcccccagggaccatatggtccgggcccgcaagggcccgggccaagaggtccaggtcctcaaggacccgggccccaaggaccaggaccacaggggccctcaggtccggggccgcaaggaccgtacggacccggcggagtgagcgtggtttctgcctccgtatcgggacctggcccgcaaggtccctctggtccc

SEQ ID NO: 14 - DNA, 1744 bp, Bombyx mori
gggagaaagcatgaagtaagttctttaaatattacaaaaaaattgaacgatattataaaattctttaaaatattaaaagtaagaacaataagatcaattaaatcataattaatcacattgttcatgatcacaatttaatttacttcatacgttgtattgttatgttaaataaaaagattaatttctatgtaattgtatctgtacaatacaatgtgtagatgtttattctatcgaaagtaaatacgtcaaaactcgaaaattttcagtataaaaaggttcaactttttcaaatcagcatcagttcggttccaactctcaagatgagagtcaaaacctttgtgatcttgtgctgcgctctgcaggtgagttaattattttactattatttcagaaggtggccagacgatatcacgggccacctgataataagtggtcgccaaaacgcacagatatcgtaaattgtgccatttgatttgtcacgcccgggggggctacggaataaactacatttatttatttaaaaaatgaaccttagattatgtaacttgtgatttatttgcgtcaaaagtaggcaagatgaatctatgtaaatacctgggcagacttgcaatatcctatttcaccggtaaatcagcattgcaatatgcaatgcatattcaacaatatgtaaaacaattcgtaaagcatcattagaaaatagacgaaagaaattgcataaaattataaccgcattattaatttattatgatatctattaacaattgctattgcctttttttcgcaaattataatcattttcataacctcgaggtagcattctgttacattttaatacattggtatgtgattataacacgagctgcccactgagtttctcgccagatcttctcagtgggtcgcgttaccgatcacgtgatagattctatgaagcactgctcttgttagggctagtgttagcaaattctttcaggttgagtctgagagctcacctacccatcggagcgtagctggaataggctaccagctaataggtagggaaaacaaagctcgaaacaagctcaagtaataacaacataatgtgaccataaaatctcgtggtgtatgagatacaattatgtactttcccacaaatgtttacataattagaatgttgttcaacttgcctaacgccccagctagaacattcaattattactattaccactactaaggcagtatgtcctaactcgttccagatcagcgctaacttcgattgaatgtgcgaaatttatagctcaatattttagcacttatcgtattgatttaagaaaaaattgttaacattttgtttcagtatgtcgcttatacaaatgcaaacatcaatgattttgatgaggactattttgggagtgatgtcactgtccaaagtagtaatacaacagatgaaataattagagatgcatctggggcagttatcgaagaacaaattacaactaaaaaaatgcaacggaaaaataaaaaccatggaatacttggaaaaaatgaaaaaatgatcaagacgttcgttataaccacggattccgacggtaacgagtccattgtagaggaagatgtgctcatgaagacactttccgatggtactgttgctcaaagttatgttgctgctgatgcgggagcatattctcagagcgggccatacgtatcaaacagtggatacagcactcatcaaggatatacgagcgatttcagcactagtgctgcagtc

SEQ ID NO: 15 - DNA, 1205 bp, Bombyx mori
ggaacaggctcttctggatttggaccatatgtagcaaatggcggatatagcagacgtgaaggctacgaatacgcttggtcgtcaaaatctgactttgaaactggaagcggtgctgcctctggtgctggtgctggtgctggttcaggtgctggtgctggttcaggtgccggtgctggttcaggtgctggtgctggttcaggtgccggtgctggtggtagcgtcagttacggagctggcaggggatacggacaaggtgcaggaagtgcagcttcctctgtgtcatctgcttcatctcgcagttacgactattctcgtcgtaacgtccgcaaaaactgtggaattcctagaagacaactagttgttaaattcagagcactgccttgtgtgaattgctaatttttaatataaaataacccttgtttcttacttcgtcctggatacatctatgttttttttttcgttaataaatgagagcatttaagttattgtttttaattacttttttttagaaaacagatttcggattttttgtatgcattttatttgaatgtactaatataatcaattaatcaatgaattcatttatttaagggataacaataatccatgaattcacatgcacatttaaaacaaaactaaattacaataggttcatataaaaacaacaagtatgccttctcaactaagaatactatattgtttaaaccgtaaaaaaagtcatatttctgtatatcaaaacacatctaatattaaaaaaacagtcagcaagcacttacaagtgtgggctcggacagcaattacctggtctcaggagacacttgaagaacgagaagcacgtctctctgtcgattgcgaggctcatgcactatcgcttgagtctgagacctttactgatagggaaatccgtttgagctctcagagggttcggacagcaaaagcttgcccggtctcaggagacattagaagaacgggaagcataactcaataccgatcgcgtttccattgagcctatgcttcgtgataataataaataaagcccaaggtcagacgctaaaagttgtgggtgtagatataagtgttaactgcttttcacacagtcaactcggtcacgcggttatgtagctctgtcacatgctagcaatcctagcaacctgtatgctttgatccccgatggctatacgtctacgtcgtgtatagagaagctt

SEQ ID NO: 16 - DNA, 2970 bp, Homo sapiens
ggtccacccgggccccctggaccaccggggccacctggactgggcgggaattttgcgccccaattgtcatatggttatgacgagaagagcacgggaggaatatctgtcccagggccgatgggacccagtgggccccgtggattgccaggtcccccaggggctccaggcccccaggggttccaggggccacctggggagcccggcgagcctggagcgagtgggccgatgggaccaagaggcccacctggaccccccggaaaaaacggagacgatggcgaggcaggaaagccggggcgacccggcgagcgtggtccccccggaccccagggagctcgtgggctgcccggcactgcaggtttaccagggatgaaaggccatcgaggattttctggattagacggtgctaagggcgacgctgggcccgcaggccctaaaggggagcccggcagccctggtgagaacggagcgccagggcaaatgggtcctagaggcctaccgggtgagcgaggaagacctggcgctccggggcctgctggaaatcccggggcggacgggcaacccggtgcaaaaggtgcaaatggtgcgcccggaatagctggcgcgcctggtttcccgggggcacgtgggccgagcggaccgcagggtcctggtggtccgccgggccctaaaggaaactccggagagccaggcgcaccagggtcgaagggggataccggcgcgaagggagaacctgggccggtaggggtacaggggccacctggccccgcaggggaggaaggtaagagaggtgctagaggagagcccggtcctacaggcctccccggcccacccggtgagcgtggaggcccgggaagtagaggtttccccggtgctgacggcgttgccgggccaaagggtccggctggtgagcgagggtcacccggacccgcagggcccaaaggctcgcccggagaggccgggcgcccaggtgaggctggcctgcctggagcgaaagggctgacgggatccccggggagcccgggaccggacggaaaaacaggacccccgggaccagccggccaagacgggcgacccggccctcctgggcctcctggcgcgcgaggtcaagccggagtcatgggctttccgggtcccaagggtgccgctggagagccaggcaaagcgggcgaacgtggcgtgccaggaccaccgggtgcagttggtcctgcaggtaaagatggtgaggcaggggcacagggtccgcccggtccagctggccctgcaggtgagcgaggggagcaggggccagctgggtctcctggatttcaaggattgcccgggccagccggaccgccgggcgaagccggcaaacccggcgaacagggggtgccaggggacttaggtgctccgggaccctcgggggcgcgaggagagagaggttttccgggtgagcgaggtgttcaaggaccacccggtccagcaggcccaaggggggccaacggagcaccagggaatgatggtgcgaagggcgacgccggggccccgggtgcgccgggaagtcagggagcgccgggcctgcaaggtatgccgggagagcgtggggcggctggattgccgggtcctaagggcgatcgcggcgacgctgggccaaagggtgccgatggaagtcccggcaaagatggagtacgtgggttgactggtcccattggcccgccggggccagcgggtgcgcctggagacaagggagaatcgggtccttccgggccggcgggtcccacaggggcgagaggtgcaccgggggaccgcggcgaaccgggtccacctggcccagctgggttcgctgggccccccggggcggacggtcaaccaggagccaagggagagccaggcgacgctggcgccaagggagatgcggggccccccggacctgccgggcctgcagggccccccggacctataggaaatgttggtgcccccggggcaaaaggcgcgcgcgggtccgcgggcccgccaggggcgacgggttttcccggggccgctggaagggtgggcccacccggaccttccggaaacgccgggcctccaggaccgcctggtcctgcggggaaagaaggaggaaaagggccacgcggagaaacgggcccagcgggaagaccgggagaagttggaccacctggtcctccgggccccgctggggagaaaggaagcccaggcgctgacggaccggcgggtgcgccaggaacacccggacctcaaggcatagcaggccaaagaggagttgttgggctgccgggtcagagaggagaacgaggctttcctggcctgcccggcccttcaggcgagcccgggaaacagggcccttcaggggcatctggagaaaggggcccgccaggacctatgggtcccccggggctagccggacctccgggcgaatcaggcagggagggagcgcccggggccgaaggttcgccggggcgcgacggatcgccaggagctaaaggggaccgaggtgaaactggaccagcgggccctccgggggcaccgggtgctccgggcgccccggggcctgtggggcccgcagggaagtccggtgaccgaggtgagactggcccagcgggcccagctggaccagtcggtcctgtcggggcccgtgggcccgcaggaccccaaggccctagaggggataaaggcgaaaccggggagcagggagaccgagggatcaaaggacataggggctttagtggtctgcaagggccaccaggtccccccggttctcctggggagcagggcccctctggggccagcggcccagctggcccccgaggccccccaggatctgccggtgcaccggggaaggacggtcttaatgggttgccagggcccatcggacccccgggcccgcgtggacgcaccggagacgccggcccagtaggcccgcccggcccgcctggtccgcctggtccgccgggccccccttcggcagggtttgac

SEQ ID NO: 17 - DNA, 1893 bp, Homo sapiens
ggtgcgggtctcggggcgctaggtggaggagctttgggcccaggtgggaagccgctaaagcctgttccaggtggccttgcaggggcaggccttggtgcgggtctcggagcctttcctgcggttacgtttccaggagcactggtgccgggaggcgttgccgatgcggcggccgcttataaagccgctaaagcgggcgctggattagggggcgttcccggcgtgggtggattaggtgtctcagcaggagctgtggtgccacagcctggtgcaggtgtgaaacccggcaaagttcccggcgttggtttaccaggtgtgtatcccggaggagtcttgcctggagcccgttttcccggagtcggagtcctcccaggtgtccctaccggcgcaggggtaaagccgaaagctcctggtgtgggtggagccttcgcaggcattcccggggtaggcccgttcggtggacctcagccgggtgtgccacttggttaccccattaaggcaccgaaactcccaggggggtacggactaccatataccacgggcaaacttccctatggatatggaccaggaggcgtagctggcgccgctggtaaagctggttatccaaccggcacaggggtcgggccccaagccgcggcagctgcggccgcaaaagcggctgcaaaattcggcgcgggagcagctggggtgcttcctggggttggtggtgcgggtgtacctggggtaccaggtgcgatacctgggatagggggcatagccggggttggcactcccgcagcggctgccgctgcagccgccgctgctaaagcagcaaaatatggcgccgcggcagggcttgtgcctggcggacctggctttggccccggggttgtaggtgttcccggtgccggggtccccggcgtcggtgtacctggagctggaataccggtcgtgccaggtgcaggcattcctggtgctgcagtacctggtgtagtatcccccgaggcagcggcgaaggctgctgccaaagcggccaagtacggcgcgcgcccaggagttggggtaggaggcatccccacctacggcgttggggcgggagggttcccgggttttggcgtgggagtaggtggaattcccggagtcgcgggtgtacccggcgttggaggtgtgcccggcgtgggaggggttcccggagtagggatttctcccgaggcacaggctgctgctgcagctaaagcagccaagtacggcgtcgggactcccgccgctgcagccgcgaaagcggcagccaaagcggcacagtttggcctagtccctggtgttggagttgcccctggggtgggcgttgccccgggagttggcgtggctcccggagttggcctcgccccgggcgtcggtgtcgcgccgggagtcggtgttgcgcccggcgtgggggtcgcccccggcattggtcccgggggagtagcggcagcagccaaatccgcagctaaggtagcggcgaaagcccaactgcgagcagcggcaggcttgggtgctgggatacccggcctgggcgtaggcgtaggcgtacctggcctgggggtaggagcgggtgtgcctggcctgggcgtcggagctggtgtacccggattcggagcagtcccaggcgctctagctgctgctaaagcagccaagtacggggcggcggtgccgggagtactaggagggttgggggcactcggcggggtaggaattccgggtggagttgttggagcggggcctgctgccgccgctgcagcggccaaagctgcagcaaaagcagcacaatttggactggttggggccgccggactcgggggactgggagttggcgggcttggagtccccggggttggtggacttggtggaatacctccggccgcggcagctaaggcggcc

SEQ ID NO: 18 - DNA, 1419 bp, Homo sapiens
atgacgacgtgttctcgtcaatttacaagcagtagctctatgaagggctcctgcggaattggaggcgggatcggtggagggtcgtcccgcatctctagtgtcctcgctggaggatcttgtagggcgccttctacttacggtggcggtctatccgtgtcatcgcgcttttctagtggcggcgcatgcggcctaggagggggctacggcggcggtttctcatccagcagcagctttggctctggtttcgggggaggctacgggggtggtttaggtgcaggcttcggtggaggactgggcgccggatttggtggcgggttcgcggggggtgatggtctcttggtcggatccgagaaagtgactatgcagaacctaaacgatcgtcttgcctcatacctcgacaaagtccgtgccttggaggaagccaatgcggatctcgaggttaaaataagggattggtatcaacgccagcgtccttctgaaataaaggattattccccgtactttaagacaatcgaagacctaagaaacaagattattgcggctactatagagaatgctcagcccatacttcagatcgacaacgcccgcctcgctgctgatgacttcagaacgaagtacgagcatgaattggccctaaggcaaacagtcgaagctgacgttaacggactgcgcagagtgttggacgagttaaccttagcacgaactgatttggagatgcaaattgagggcttaaaggaagaactagcttatttgcgtaagaatcatgaagaagagatgttagcgttacgcggccagaccggaggggatgttaacgtggaaatggatgccgcaccgggcgtagatctcagtaggatcttgaatgagatgagagaccaatacgaacaaatggccgaaaagaataggcgtgatgcggaaacatggtttctatcaaagacggaagagctgaataaggaggttgcctcgaatagcgagcttgtacaatcgagcagaagtgaggtcacggaactgcgtagagttctgcagggtttagaaattgagttgcaatcacagttgtcgatgaaggcgagtctggaaaacagcctggaggaaacgaaagggcgctactgtatgcaactctcacaaattcagggtttaatcggtagcgtagaagagcagctggcgcagctacgctgcgaaatggagcagcaatctcaggagtatcagatattattggatgtcaagactcgcctcgagcaagagatagcgacttaccgccgattgctagaaggagaagacgcgcacctctcaagtcaacaagcgagcggccagtcttactcttcacgtgaggtcttcactagcagctcttcgagctcatcgcgacaaacgcgcccgattttgaaggaacaatcatccagtagcttttcacaaggacaaagttca

SEQ ID NO: 19 - DNA, 3348 bp, Rhyacophila obliterate
tcagaatccgaatctgattcggacagcagcgaggactccgggagcaccgaaaaaattataaaacagatcataattgttcaagagaagcccaagcatggacatcatcatgccaaagagaagatctacgaagaggagattattattaagaagataggagatttacccaaaaaagactgtgatgaaaataagccgagggaagtcacctcatggcgccacactcttccgaacagaaagagtatatcgataagtaagtctattagtatagagcaaattataaagcctcacgttataacccgaataagaacatccaaaagcagcagtctaagtataagtgtcgagcgacctaggcgaattatatcacctataatttcgggaggatggaacaggcacaaaccaaggaaatattcggccagttcctccatttctaagagtattagtattgagagagttattacacctgcagtatggactaggattcacaagtcggtgagcgtcagccatagcgtgtcagttgagcaccgcaggagaatagcacctattgtggtggactactcgatctcctcatcattatcaatatcaggtgaggggcgaggattgggtagagggaagcatggttggggggggcttggccacggcggattgggtggccttgggcatggaggtttaggtggcctgggccatggtggactgggtggactcggtcacggtggcttgggtggtctcggctccgattcaggcgacctaagtatctctagtagccttagtattagcggtgagggacgaggcctcgggcgtgggaaacacggttggggtggacttggccacggcggtttaggaggattgggccacggagggttgggggggttgggccatggaggccttggtggcctgggccacggaggactgggggggctaggctcagattcgggtgacctaggcggaatcggtggcctactaggacccggagggcgccgtggctggggtcgtcgcggaccgggcaaatactcagcatcagtttcaatctcacactctatcagcatagaacgtgtgatcacacccggggtttatacaagcattcaccgatctgtttccgtaagtcactcggtgagtgttgaacataggcgcaggattgcgcctatagtagtcgactactcaatctcgtcgtcgctgtctatctcgggtgaagggcgaggcttgggacgcggtaaacatggctggggcggtttaggccatggaggacttggaggattagggcacggcggtttagggggtctaggacatggaggattggggggcttagggcatgggggacttggcggattaggctcagacagcggcgatctaggcgggattgggggccttttgggccctggggggcgcagaggttggggaaggcgaggcccaggtaaatattcggcttcggtatccattagtcattcaatttcgatagagcgagtaatcacgccgggggtatatacatccattcaccgaagtgttagcgtatcacattctgtaagcgtcgagcataggcgcagaatcgcacccatcgttgtggactattcgatatcgtccagcctctcgatatcaggcgaaggccgtggtttgggacgaggcaagcacggttggggggggttaggccacggaggcctgggtgggttaggacatggtggactcggcggcctagggcatggaggacttggcggtttgggacacggcggcctgggcggcctagggtcggattctggagacctgggcggcataggtggtttgctaggacctggtggccgcaggggctgggggagaagaggtcctggcaaatacagcgcgtccgtgtcgataagccatagcattagcatagagagggtcatcacaccaggggtctacacctcaatacacagaagtgtaagtgtgtctcactcagtttccgttgagcaccgcaggcgtattgcgcccatcgtagtagactatagtatctcgtcgtcgctatctatttccggggaggggaggggcctcggaagaggcaaacacggttgggggggtttgggtcatggcggcctaggaggtcttggacatggtggattaggaggactcgggcacggaggcctaggggggttaggacatggaggccttggaggcctgggatccgatagtggagatttaggcggaatcggaggcttgctgggccctggggggcgccgaggctggggacgcagagggcctgggaagtatagtgcgtcagtgagtatctcgcattctatatcgatagaacgagttatcactccaggagtgtatacgtcaatacaccgaagtgttagtgtctcgcattcggtatctgtcgaacacagacgccgtattgcgcctatcgtggttgattattcgattagtagctcgctatctatatccggcgaaggtcgcggcctcggtagagggaaacatgggtggggtggtctagggcacggtgggttaggaggcctcggccacggggggctaggtgggctggggcatggaggtctgggtgggttggggcatggtggtcttggtgggctcggctcggattcgggggatcttggaggcataggcggattactcggtcccggagggagaagaggctgggggaggcgcgggcccggcaaatactcggcatcagtctcaatctctcactcaatctctatcgagcgcgttattaccccgggcgtgtatacgagtatacataggagtgtgtccgtatcgcactcagtgtctgtggaacacagacgacgaatcgcacctatagtcgttgactacagtatctccagttcattatcgatttccggggagggacgaggcttgggacgagggaagcacggatggggcggacttggtcatggtgggctagggggtctcgggcatgggggattgggtggattgggacatggtggcttagggggactcggccacggggggttggggggcctcgggtctgatagtggcgatttagggggaatcggtggtctcctaggccccggtgggcgaaggggctggggacgccgcgggcctgggaaatactctgcttctgtttccatctctcattcgatatctatcgaacgtgtaatcacgcccggagtgtatacatctatacatagaagtgtcagcgtcagccatagtgtaagtatagaaagaccaaggaggattatctctccggttgttgattattctctatcaattagtgccagttcttcagtttccgctgaagggggtcgccgtcgtcgtttacccggttcgtgcggcactatcataagtgacagctcgagtaacgttggcggtctggggcgtataggggcgttacacaaattgggcggtgggcgcatt

SEQ ID NO: 20 - DNA, 279 bp, Unknown
  SspDnaX N-terminal sequence
tgcctgaccggcgatagccaggtgctgacccgcaacggcctgatgagcattgataacccgcagattaaaggccgcgaagtgctgagctataacgaaaccctgcagcagtgggaatataaaaaagtgctgcgctggctggatcgcggcgaaaaacagaccctgagcattaaaaccaaaaacagcaccgtgcgctgcaccgcgaaccatctgattcgcaccgaacagggctggacccgcgcggaaaacattaccccgggcatgaaaattctgagcccggcg

SEQ ID NO: 21 - DNA, 120 bp, Unknown
  SspDnaX C-terminal sequence
ccgcagtggcataccaactttgaagaagtggaaagcgtgaccaaaggccaggtggaaaaagtgtatgatctggaagtggaagataaccataactttgtggcgaacggcctgctggtgcat

SEQ ID NO: 22 - DNA, 333 bp, Unknown
  Cth-Ter N-terminal sequence
cagctggcgctggataccccgattccgaccccggatggctggaccaccatgggcgaaattaaagcgggcgataaagtgattgatgaaaaaggccgcccgtgcaacgtggtggcgattagcgaaattgatgataccgaacaggcgtataaaattaactttcgcgatggcaccagcattgtggcgggcgaacgccatctgtggaaagtgcaggtgaccaacaacggccgccgcgaaaaactgctgaccaccggcgaaatgtatcagaaacagtttaaaaccaaaagcaaagaaaaccgcgcgctgtttcgcattccgattgcggatgcgtttatt

SEQ ID NO: 23 - DNA, 120 bp, Unknown
  Cth-Ter C-terminal sequence
agccattttcattatattaaaagcattgaaaaaaccggcaaaaccaaaatgcgctgcattcaggtggatagcccgagccgcctgtatctggcgggcaaaagcatgattccgacccataac

SEQ ID NO: 24 - DNA, 318 bp, Unknown
  Mp-M-DnaB N-terminal sequence
gcgctggatgtggaaaccccgattctgaccggcaacggctggaaaaaaatgggcgatattcaggtgggcgattatgtgcatgcggcggatggcaccctggcgcgcgtgagctatgtgagcgaacgccattggcgcgattgctttagcgtgcagtttgcggatggcgcggaactggtggcgagcgatcatcatctgtgggcggtgaacgatcgcctgaaaggcgaacgcgtgattgataccgcggaactgtatcgcacccagacctatggcgcgcgcggcgatcgccgctataccgtgaccgtgccggaagcgctggat

SEQ ID NO: 25 - DNA, 120 bp, Unknown
  Mp-M-DnaB C-terminal sequence
gcgcgcaccaacaccattaccagcgtgaccccggtgccgaccgtggaaaccgtgtgcattcagattgatcatccgagccatgtgtttctggcgggcaaaagcctgaccccgacccataac

SEQ ID NO: 26 - DNA, 402 bp, Unknown
  TvoVMA N-terminal sequence
tgcgtgagcggcgaaaccccggtgtatctggcggatggcaaaaccattaaaattaaagatctgtatagcagcgaacgcaaaaaagaagataacattgtggaagcgggcagcggcgaagaaattattcatctgaaagatccgattcagatttatagctatgtggatggcaccattgtgcgcagccgcagccgcctgctgtataaaggcaaaagcagctatctggtgcgcattgaaaccattggcggccgcagcgtgagcgtgaccccggtgcataaactgtttgtgctgaccgaaaaaggcattgaagaagtgatggcgagcaacctgaaagtgggcgatatgattgcggcggtggcggaaagcgaaagcgaagcgcgcgattgcggcatgagcgaagaatgc

SEQ ID NO: 27 - DNA, 150 bp, Unknown
  TvoVMA C-terminal sequence
atggaagcggaagtgtataccagcctggaagcgacctttgatcgcgtgaaaagcattgcgtatgaaaaaggcgattttgatgtgtatgatctgagcgtgccggaatatggccgcaactttattggcggcgaaggcctgctggtgctgcat

(19) United States
(12) Patent Application Publication
(10) Pub. No.: US 2022/0017917 A1
(43) Pub. Date: Jan. 20, 2022

(54) Synthesis of High Molecular Weight Proteins Using Inteins

(71) Applicant: Kraig Biocraft Laboratories, Inc., Ann Arbor, MI (US)

(72) Inventors: Kimberly Kraig Thompson, Lansing, MI (US); Trevor Logan Kane, Dewitt, MI (US); Ana Laura Licon, Brighton, MI (US); Jimi Craig Miller, Williamston, MI (US)

(21) Appl. No.: 17/377,312

(22) Filed: Jul. 15, 2021

Related U.S. Application Data
(60) Provisional application No. 63/053,469, filed on Jul. 17, 2020.
(60) Provisional application No. 63/053,486, filed on Jul. 17, 2020.

(51) Int. Cl. C12N 15/85; C07K 14/435; C12N 15/74; A01K 67/033

(52) U.S. Cl. CPC C12N 15/8509; C07K 14/43518; C07K 14/43563; C12N 15/74; A01K 2217/052; C12N 2015/8518; A01K 2267/01; A01K 2227/706; A01K 67/0339

(57) ABSTRACT

This disclosure is directed to split intein protein production systems using transgenic target organisms such as Bombyx mori. A vector set for transforming a target organism includes: a first vector having a first donor sequence that encodes (i) a first non-native protein and (ii) at least one split intein domain; a second vector having a second donor sequence that encodes (i) a second non-native protein and (ii) at least one split intein domain. The respective split intein domains encoded by the first and second vectors are configured to associate with one another and ligate the first and second non-native proteins to thereby form a fused protein.

### CROSS-REFERENCE TO RELATED APPLICATIONS

This application claims priority to and the benefit of U.S. Provisional Patent Application Ser. No. 63/053,469, filed Jul. 17, 2020 and titled “Method of Producing Auto-Assembling High Molecular Weight Proteins” and claims priority to and the benefit of U.S. Provisional Patent Application Ser. No. 63/053,486, filed Jul. 17, 2020 and titled “Method for Creating High Molecular Weight Proteins using Auto-Assembly in Bombyx Mori”. The entireties of each of the foregoing are incorporated herein by this reference.

### INCORPORATION BY REFERENCE OF SEQUENCE LISTING

The document filed in conjunction with this application includes a numerical listing of sequences corresponding to the sequences described herein, each identified by a unique SEQ ID NO. The 21824-4-1-SeqList.txt file was created on Jul. 15, 2021 and has a size of 76,008 bytes. The 21824-4-1-SeqList.txt file is expressly incorporated herein by this reference.

### BACKGROUND

### Technical Field

This disclosure generally relates to methods of producing high molecular weight proteins using split intein tags and a microbial protein production system or a transgenic Bombyx mori (i.e., domestic silkworm) protein production system.

Related Technology

Bombyx mori is an insect from the Bombycidae moth family, most commonly referred to simply as the silkworm (in the larvae stage) or silk moth (in the adult stage). Bombyx mori were domesticated thousands of years ago in China for their ability to produce relatively large quantities of silk. Selective breeding has, over time, enabled domestic Bombyx mori to produce almost 10 times as much silk as their wild counterparts.

After a silkworm has molted four times, it will enter the pupal stage by forming a cocoon made of raw silk. The cocoon is typically formed from a single filament that can average more than 900 meters in length. The silk is harvested by steaming or boiling the cocoon before the adult moth can form and release protease enzymes, which would damage the silk of the cocoon.

Bombyx mori silk is made up of two major components: fibroin and sericin. Fibroin is produced in heavy chain, light chain, and glycoprotein P25 forms. When the silkworm produces silk, the heavy and light chains are linked by disulphide bonding, and the P25 integrates via non-covalent interactions. The sericin proteins are hydro-soluble and function to coat and adhere separate fibroin filaments as the silkworm generates the silk. Sericin proteins are glue-like proteins that coat the heavy chain and light chain proteins and allows neighboring silk threads to adhere together.

There have been attempts to produce transgenic silkworms capable of expressing non-native proteins, particularly spider silk proteins. However, thus far it has been challenging to transgenically produce spider silk with the desired mechanical characteristics, at appropriate scale, and in a cost-effective manner. Other protein production systems such as those that utilize microbes (e.g., E. coli or yeast-based systems) are limited by the inability of the microbes to handle large proteins and/or proteins associated with repetitive DNA sequences. Examples of such proteins include spider silk proteins, collagen, elastin, and other fibrous proteins. Accordingly, there are a number of disadvantages with conventional protein production technology.

### SUMMARY

As discussed above, it has been challenging to produce spider silk at large scale and in a cost-effective manner in part due to the inability to culture spiders en masse for this purpose. Moreover, although there have been attempts to utilize other organisms to produce spider silk and other large, medically and industrially relevant proteins, these efforts have also met significant challenges such as the inability of microbial systems to generate large proteins that include several repeating structural motifs, the need to purify the intended product, and/or difficulty in achieving an end product with the desired mechanical properties.

Silkworms transformed with DNA encoding spider silk proteins or DNA encoding other proteins of interest is one promising approach to achieving effective and economical production of enhanced protein products. Silkworms have the inherent ability to spin fibers at relatively high purity levels, reducing the need for complicated downstream processing of the product. Silkworms have also been cultured for thousands of years, and a mature sericulture industry is already in place.

Embodiments disclosed herein are directed to methods of genetically modifying a target organism to enable protein production using a split intein system. The target organism may be a microbe (e.g., bacteria such as E. coli, Bacillus, Actinomycetes, Pseudomonas, or lactic acid bacteria, or yeast such as Saccharomyces cerevisiae, Pichia pastoris, or Schizosaccharomyces pombe). Alternatively, in certain preferred embodiments the target organism is Bombyx mori.

Certain embodiments are directed to vectors configured to be used in conjunction with one another to enable production of one or more proteins by way of an intein system. In one embodiment, a vector set includes: a first vector having a first donor sequence that encodes (i) a first non-native protein and (ii) at least one split intein domain; a second vector having a second donor sequence that encodes (i) a second non-native protein and (ii) at least one split intein domain. The respective split intein domains encoded by the first and second vectors are configured to associate with one another and ligate the first and second non-native proteins to thereby form a fused protein.

Other embodiments include additional vectors with donor sequences encoding further non-native proteins and intein domains. For example, some embodiments include a third vector with a third donor sequence that encodes for a third non-native protein, and the split intein domains function to ligate the first, second, and third non-native proteins to form a fused protein. Other embodiments may further include a fourth vector that similarly functions to enable a fourth non-native protein to be ligated to the third non-native proteins. Other embodiments may further include even more of such vectors (e.g., fifth, sixth, seventh, etcetera) that function to enable ligation of even more non-native proteins to form a fused protein.

Certain embodiments disclosed herein include plasmid constructs for molecular cloning of donor sequences, and/or for transformation of a target organism with the donor sequences. Certain embodiments described herein are directed to transgenic organisms that have been transformed with such donor sequences and that can produce one or more non-native proteins (e.g., fibrous proteins such as spider silk, collagen, elastin, keratin, fibrin, or other medically and/or industrially relevant proteins). Certain embodiments described herein are directed to the protein product produced by such transgenic organisms.

The split intein systems described herein beneficially enable the production of proteins that combine multiple sub-components (i.e., multiple pre-fused proteins) to form beneficial fused proteins with enhanced mechanical properties and/or other beneficial properties. In certain embodiments where the result is a fibrous product, for example, the resulting protein product can provide enhanced strength and elasticity.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1A schematically illustrates exemplary pre-fused proteins each having one or more inteins attached thereto configured to enable fusion of the pre-fused proteins;

FIG. 1B schematically illustrates a precursor protein resulting from the fusion of multiple pre-fused proteins by way of interaction between the respective inteins;

FIG. 1C schematically illustrates a fused protein resulting from the split intein reaction after the inteins have been excised; and

FIGS. 2A-2C illustrate additional examples of precursor proteins that can be synthesized using the set of pre-fused proteins of FIG. 1A.

### DETAILED DESCRIPTION

Overview of Transgenic Bombyx mori

Bombyx mori silk is made up of two major components: fibroin and sericin. Fibroin is produced in heavy chain, light chain, and glycoprotein P25 form. When the silkworm produces silk, the heavy and light chains are linked by disulphide bonding, and the P25 integrates via non-covalent interactions. The sericin proteins are hydro-soluble and function to coat and adhere separate fibroin filaments as the silkworm generates the silk. In commercial silk production, the sericin is typically removed as an unimportant side product.

Although the fibroin heavy chain and the fibroin light chain are included in the silk in approximately the same molar ratio, the fibroin heavy chain has a much higher molecular weight than the light chain (about 350 kDa compared to about 26 kDa). The fibroin heavy chain thus makes up the majority of Bombyx mori silk. Targeting the fibroin heavy chain gene (FibH) for modification therefore allows significant changes to the mechanical properties of the silk generated by the resulting transgenic silkworm.

As used herein, “modification” of the fibroin heavy chain gene includes embodiments where the entire FibH gene, or one or more portions thereof, are knocked out of the silkworm genome and replaced with a knock-in insert (e.g., using a truncation vector). The term also includes embodiments where one or more inserts are inserted at a position within the FibH gene and/or at a position functionally adjacent to the gene (e.g., using an insertion vector). Some knockout embodiments are configured to knockout exon2 (about 16 kbp) of the FibH gene. In some knockout embodiments, at least about 50% of the FibH gene is knocked out, or at least about 60%, or at least about 70%, or at least about 80% of the FibH gene is knocked out.

In some embodiments, the FibH gene is targeted in a manner that retains the native FibH promoter within the genome. As a result, the resulting transgenic silkworm is able to utilize the native promoter for expression of the knocked-in insert. Alternative embodiments target the FibH promoter for inclusion in the knockout portion and include one or more separate promoter sequences as part of the knock-in insert.

A relevant section of the FibH gene is provided as SEQ ID NO:1. Minor variations of the gene can occur due to differences in particular silkworm varieties, and the disclosed sequence is exemplary only. The skilled person will understand that the principles and components described herein can be utilized with other variations of the FibH gene with only minor or no modification required.

Exemplary DNA targets associated with specific locations of the FibH gene for targeting by guide RNAs (gRNAs) are provided as SEQ ID NO:2 to SEQ ID NO:8. In particular, SEQ ID NO:2 and/or SEQ ID NO:3 may be utilized as targets for upstream gRNAs, and SEQ ID NO:4 and/or SEQ ID NO:5 may be utilized as targets for downstream gRNAs, in a Mad7 system in which the PAM sequence is YTTN. In another embodiment, SEQ ID NO:6 and/or SEQ ID NO:7 may be utilized as targets for upstream gRNAs, and SEQ ID NO:8 may be utilized as a target for a downstream gRNA, in a CRISPR/Cas9 system in which the PAM sequence is NGG. The locations of the FibH gene in which these exemplary gRNAs targets can be found by matching the gRNA target locations (or in most instances, their reverse complements) to the corresponding location in the FibH gene (e.g., as provided by SEQ ID NO:1). In some embodiments, reverse complements of one or more of the foregoing may additionally or alternatively be utilized as suitable gRNA targets.

The particular portions of the FibH gene targeted for knockout and/or as an insertion site will vary somewhat depending on the particular gene editing process utilized. This is a result of inherent differences in gene editing techniques. For example, the standard Cas9 nuclease requires a protospacer adjacent motif (PAM) with sequence NGG, whereas the standard Mad7 endonuclease requires a PAM with sequence YTTN. Other Cas9 or Mad7 type nucleases, or other gene editing nucleases may have other associated PAM sequences, and thus the corresponding gRNAs may be varied accordingly. Other gene editing techniques such as those using transcription activator-like effector nucleases (TALENs) or zinc finger nucleases (ZFNs) have other inherent characteristics that must be accounted for when selecting a particular target site within the FibH gene. The skilled person, in light of the teachings of this disclosure, is able to determine appropriate FibH targets for these other gene editing processes.

### Exemplary Donor Inserts

As mentioned above, many spider silks provide superior mechanical properties and would be beneficial for a variety of applications. However, cost-effective and appropriately scaled production of such silks has been elusive due to the technical challenges involved with producing the silk. The vectors and related methods described herein include spider silk protein sequences that enable the resulting transgenic silkworms to produce an enhanced silk product with beneficially enhanced mechanical properties.

Examples of sequences encoding spider silk proteins that can be included in the donor insert include those related to the proteins MaSp2, flagelliform, A2S8 (which includes alternating repeating motifs of MaSp2 and flagelliform), MaSp1, MaSp4, and MiSp. Although these proteins are presently preferred, other spider protein sequences may additionally or alternatively be included. Particularly preferred sequences are those associated with tangle-web weaver spiders (e.g., Latrodectus hesperus) and orb-weaver spiders such as golden orb-weaver spiders (Nephila) and the Darwin's bark spider (Caerostris darwini). These types of spiders can produce silk with extremely beneficial mechanical properties.

In some embodiments, a donor insert encodes a single protein. In other embodiments, the donor insert includes sequences that encode two or more proteins. For example, a donor insert may include a set of sequences that encodes two or more spider silk proteins such as A2S8, MaSp1, and/or MaSp4. Donor inserts may additionally or alternatively include repeated sequences that would each separately encode the same protein. For example, a sequence that encodes for a particular protein may be repeated multiple times within the insert in order to provide a translated protein that is multiple times longer than the native protein, thereby providing desired differences in mechanical properties.

An example of an effective sequence that encodes for A2S8 is provided by SEQ ID NO:9. The A2S8 protein is a combination of alternating repeating motifs of MaSp2 and flagelliform that beneficially provides effective strength and elasticity.

Examples of effective sequences that encode for MaSp1 are provided by SEQ ID NO:10 (Caerostris darwini), SEQ ID NO:11 (Latrodectus hesperus), and SEQ ID NO:12 (Nephila clavipes). An Example of an effective sequence that encodes for MaSp4 is provided by SEQ ID NO:13 (Caerostris danvini).

In some embodiments, the donor insert can further include a 5′ homology arm and/or a 3′ homology arm to promote integration with the host genome. The homology arms can optionally include respective N-terminal domain (NTD) and C-terminal domain (CTD) sequences. The NTD and CTD are native sequences of the silkworm FibH gene. The inclusion of the NTD and/or CTD promotes better association of the translated protein with other Bombyx mori proteins. In particular, the NTD and/or CTD sequences enable the translated protein to better integrate with the light chain fibroin and the P25 proteins, which is beneficial in certain applications where such integration is desired. In some embodiments, the target for nuclease activity is located within or downstream of the native NTD. In such embodiments, an NTD included as part of the donor sequence can be utilized to further guide the donor sequence and ensure that the remaining native portions of the FibH gene stay in frame with the inserted donor sequence.

In other embodiments, the NTD and/or CTD sequence is/are intentionally omitted from the insert. Although there are certain benefits to their inclusion, they are part of the native silkworm FibH gene, and omitting them allows for the generation of a protein product with a higher proportion of non-native protein. Thus, in some applications where higher purity and higher proportion of non-native proteins are desired, omitting one or both of the NTD and CTD sequences is beneficial. Where the NTD and/or CTD are omitted, the target sites for knockout of the FibH gene can be tailored such that only the minimal required portions of the native NTD and CTD remain in the genome.

An exemplary 5′ homology arm is provided by SEQ ID NO:14. An exemplary 3′ homology arm is provided by SEQ ID NO:15. These sequences may be varied to some extent based on the particular Bombyx mori variants utilized, through modification of end portions that transition between the terminal domain sequences and the spider silk sequence(s), and/or through modification of end portions that transition between the terminal domain sequences and homologous arm sequences, for example.

In some embodiments, the donor DNA insert may additionally include sequences encoding for various tags and/or reporters. The spider silk may, for example, be fused with a reporter, such as luciferase, or with an N- or C-terminal epitope tag, such as FLAG, 6X-His, or other epitope tag known to those having skill in the art.

Although many of the above examples are directed to donor sequences encoding for spider silk, inserts encoding for one or more non-spider and/or non-silk proteins may additionally or alternatively be included in the vectors described herein. For example, some vectors may include donor sequences encoding for other types of fibrous proteins (i.e., scleroproteins) such as collagen, elastin, fibrin, various forms of keratin (e.g., in addition to or as an alternative to the spider silk proteins described herein), or combinations thereof. Vectors may also include donor sequences encoding for non-fibrous proteins such as proinsulin, human interferons, human growth hormones, human factor VIII, or any other medically or industrially useful protein.

In some embodiments, one or more of the donor sequences encode for human scleroproteins or other human proteins. Examples of donor sequences that encode for human scleroproteins include those that encode for a human collagen protein, a human elastin protein, and/or a human keratin protein.

An exemplary donor sequence encoding for a collagen protein is provided by SEQ ID NO:16 (Homo sapiens COL1A1; collagen alpha 1 chain isoform X1).

An exemplary donor sequence encoding for an elastin protein is provided by SEQ ID NO:17 (Homo sapiens ELN; precursor tropoelastin isoform variant 1).

An exemplary donor sequence encoding for a keratin protein is provided by SEQ ID NO:18 (Homo sapiens KRT16; Keratin, type I cytoskeletal 16).

An exemplary donor sequence encoding for Caddisfly (Trichoptera) silk is provided by SEQ ID NO:19 (Rhyacophila obliterate; RoHF). This particular example is codon optimized for Bombyx mori.

### Split Inteins & Fused Protein Products

An intein is a protein segment that can excise itself from other portions (exteins) of a protein and can join excised extein ends to form a new protein. A split intein system includes a first pre-fused protein and a second pre-fused protein, each with an intein domain located at a terminus. The respective intein domains of the pair of pre-fused proteins are configured to come together to form a complete intein. The complete intein then excises itself from the rest of the protein and ligates the excised ends of the first and second proteins together to create a fused protein. In this manner split inteins can be used to produce fused proteins of interest, especially those that are sufficiently large as to be difficult or impossible to synthesize through standard microbial protein production methods.

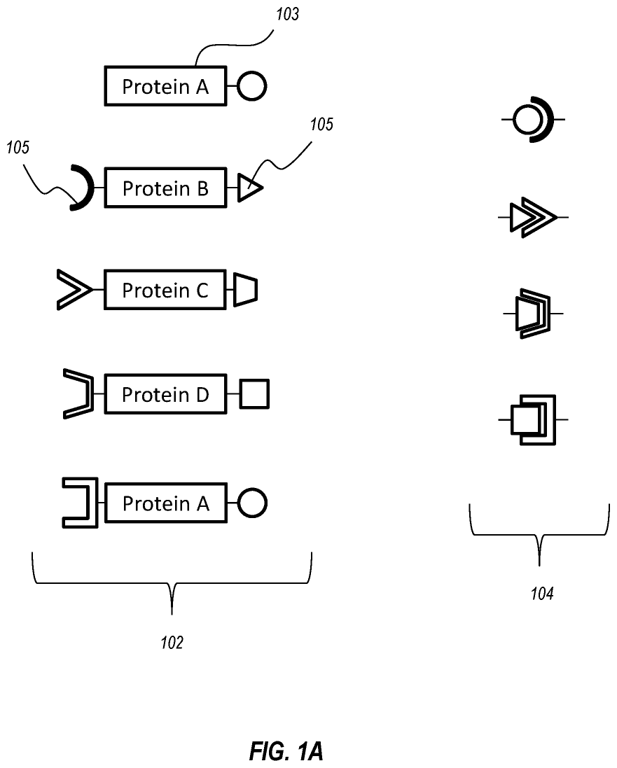

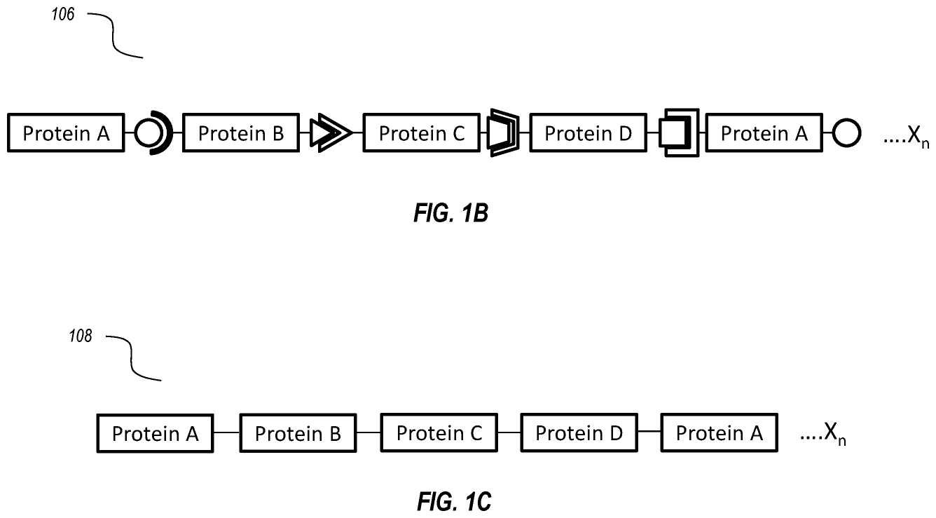

FIG. 1A illustrates multiple pre-fused proteins 102, each including an extein 103 (Protein A through Protein D in this example) and an intein domain 105 attached at one or both termini. Various complete inteins 104 are also illustrated to show how the intein domains 105 can associate as pairs to form complete inteins 104. Pre-fused proteins with two intein domains 105 have an N-terminus Int^{C} domain and a C-terminus Int^{N} domain. In FIG. 1A, the pre-fused proteins 102 with two intein domains 105 include intein domains 105 of different intein types. In other embodiments, some pre-fused proteins may include intein domains 105 from the same complete intein 104, depending on the desired protein product.

When the pre-fused proteins 102 come in proximity to one another post-translation, N-terminus and C-terminus intein domains 105 of the same type pair with one another to form complete inteins 104, thereby bringing the pre-fused proteins together to form a precursor protein. FIG. 1B schematically illustrates an exemplary precursor protein 106 made up of individual pre-fused proteins 102 that have been joined to form the precursor protein 106.

FIG. 1C schematically illustrates an exemplary fused protein 108 following auto-assembly and following self-excision of the inteins 104 from the intein-extein junctions of the precursor protein 106. Ligation of the N-terminus and C-terminus ends of the exteins 103 forms the fused protein 108. A fused protein 108 formed in this manner includes two or more exteins 103 (e.g., three, four, five, or more than five) depending on particular application needs. In some embodiments the exteins 103 may be connected by residual amino acids (e.g., about six) at the intein-extein junction as a result of the split intein reaction, but these have negligible effect on properties of the resulting fused protein 108.

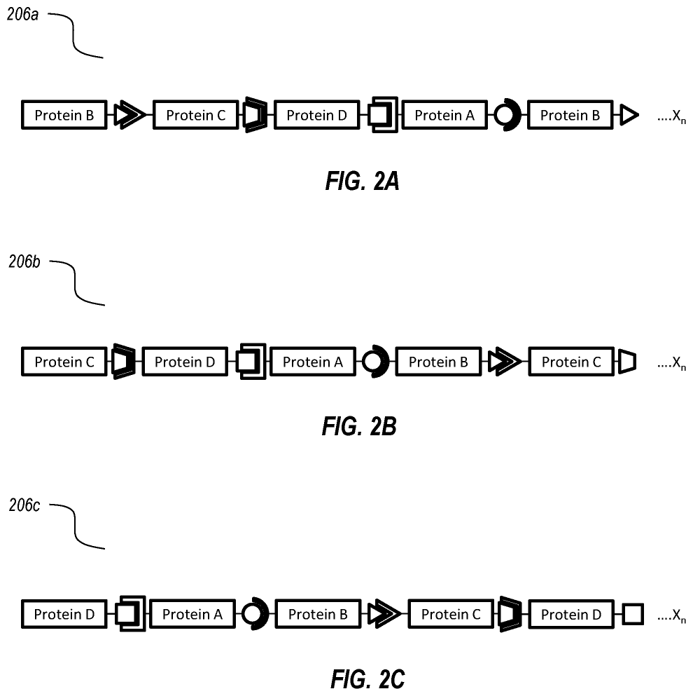

FIGS. 2A-2C illustrate additional examples of precursor proteins 206a-206c. As shown, the sequence of protein segments may be varied (e.g., may begin and/or end with a different extein 103) according to the manner in which the set of split intein reactions occur. Some embodiments may include exteins 103 that repeat consecutively. For example, one embodiment may include Protein A, followed by one or more additional instances of Protein A, optionally followed by one or more of Protein B, optionally followed by one or more of Protein C, optionally followed by one or more of Protein D, optionally followed by one or more of some other extein protein. In other embodiments, the extein sequence need not follow a particular order.

Embodiments that utilize intein domains 105 in the manner described herein may include one or more different extein proteins 103. For example, some embodiments are configured to ligate multiple instances of the same protein, while other embodiments include two or more separate extein proteins 103 configured to be ligated by way of their respective intein domains 105. Some embodiments may include more than two different extein proteins 103 configured to be ligated by way of their respective intein domains 105, such as three different extein proteins 103, four different extein proteins 103 (as in the example shown in FIGS. 1A-1C), or more than four different extein proteins 103.

The scheme illustrated by FIGS. 1A-1C and 2A-2C is useful for joining multiple different proteins from different sources (e.g., from different organisms), and is particularly useful for joining different types of fibrous proteins (e.g., different types of scleroproteins described herein). As one non-limiting example, Protein A represents MaSp1 (C. darwini), Protein B represents MaSp4 (C. darwini), Protein C represents MaSp1 (L. hesperus), and Protein D represents a human fibrous protein such as elastin.

Other combinations may also be utilized in the disclosed split intein systems. For example, some embodiments combine a spider silk protein with two or more human fibrous proteins. Some embodiments combine a human fibrous protein with two or more spider silk proteins. In some embodiments, the weight ratio of spider silk proteins to human fibrous proteins is about 0.1 to 10, or about 0.2, to 5, or about 0.4 to 2.5, or about 0.6 to 1.25, or about 0.8 to 1, or is within a range with endpoints defined by any two of the foregoing values.

Examples of split intein domain sequences that may be utilized with the embodiments described herein (e.g., that may be utilized as the inteins 104 described above) are given in Table 1 below.

TABLE 1
Exemplary Split Intein Domain Sequences

| | N Term Sequence | C Term Sequence |
|---|---|---|
| SspDnaX | Seq ID No: 20 | Seq ID No: 21 |
| S2 | tgcctgaccggcgatagccaggtgctgacccgc | ccgcagtggcataccaactttgaagaagtgga |
| | aacggcctgatgagcattgataacccgcagatta | aagcgtgaccaaaggccaggtggaaaaagt |
| | aaggccgcgaagtgctgagctataacgaaaccc | gtatgatctggaagtggaagataaccataactt |
| | tgcagcagtgggaatataaaaaagtgctgcgctg | tgtggcgaacggcctgctggtgcat |
| | gctggatcgcggcgaaaaacagaccctgagcat | |
| | taaaaccaaaaacagcaccgtgcgctgcaccgc | |
| | gaaccatctgattcgcaccgaacagggctggac | |
| | ccgcgcggaaaacattaccccgggcatgaaaat | |
| | tctgagcccggcg | |
| Cth-Ter | Seq ID No: 22 | Seq ID No: 23 |
| S2 | cagctggcgctggataccccgattccgaccccg | agccattttcattatattaaaagcattgaaaaaa |
| | gatggctggaccaccatgggcgaaattaaagcg | ccggcaaaaccaaaatgcgctgcattcaggt |
| | ggcgataaagtgattgatgaaaaaggccgcccg | ggatagcccgagccgcctgtatctggcgggc |
| | tgcaacgtggtggcgattagcgaaattgatgata | aaaagcatgattccgacccataac |
| | ccgaacaggcgtataaaattaactttcgcgatgg | |
| | caccagcattgtggcgggcgaacgccatctgtg | |
| | gaaagtgcaggtgaccaacaacggccgccgcg | |
| | aaaaactgctgaccaccggcgaaatgtatcaga | |
| | aacagtttaaaaccaaaagcaaagaaaaccgcg | |
| | cgctgtttcgcattccgattgcggatgcgtttatt | |
| Mp-M-DnaB | Seq ID No: 24 | Seq ID No: 25 |
| S2 | gcgctggatgtggaaaccccgattctgaccggc | gcgcgcaccaacaccattaccagcgtgaccc |
| | aacggctggaaaaaaatgggcgatattcaggtg | cggtgccgaccgtggaaaccgtgtgcattca |
| | ggcgattatgtgcatgcggcggatggcaccctg | gattgatcatccgagccatgtgtactggcggg |
| | gcgcgcgtgagctatgtgagcgaacgccattgg | caaaagcctgaccccgacccataac |
| | cgcgattgctaagcgtgcagtagcggatggcg | |
| | cggaactggtggcgagcgatcatcatctgtggg | |
| | cggtgaacgatcgcctgaaaggcgaacgcgtg | |
| | attgataccgcggaactgtatcgcacccagacct | |
| | atggcgcgcgcggcgatcgccgctataccgtga | |
| | ccgtgccggaagcgctggat | |
| TvoVMA | Seq ID No: 26 | Seq ID No: 27 |
| S2 | tgcgtgagcggcgaaaccccggtgtatctggcg | atggaagcggaagtgtataccagcctggaag |
| | gatggcaaaaccattaaaattaaagatctgtatag | cgacctagatcgcgtgaaaagcattgcgtatg |
| | cagcgaacgcaaaaaagaagataacattgtgga | aaaaaggcgattttgatgtgtatgatctgagcg |
| | agcgggcagcggcgaagaaattattcatctgaa | tgccggaatatggccgcaactttattggcggc |
| | agatccgattcagatttatagctatgtggatggca | gaaggcctgctggtgctgcat |
| | ccattgtgcgcagccgcagccgcctgctgtataa | |
| | aggcaaaagcagctatctggtgcgcattgaaac | |
| | cattggcggccgcagcgtgagcgtgaccccggt | |
| | gcataaactgtttgtgctgaccgaaaaaggcattg | |
| | aagaagtgatggcgagcaacctgaaagtgggc | |
| | gatatgattgcggcggtggcggaaagcgaaag | |
| | cgaagcgcgcgattgcggcatgagcgaagaat | |
| | gc | |

An exemplary SspDnaX S2 intein sequence is given by SEQ ID NO:20 (N terminal sequence) and SEQ ID NO:21 (C terminal sequence). An exemplary Cth-Ter S2 intein sequence is given by SEQ ID NO:22 (N terminal sequence) and SEQ ID NO:23 (C terminal sequence). An exemplary Mp-M-DnaB S2 intein sequence is given by SEQ ID NO:24 (N terminal sequence) and SEQ ID NO:25 (C terminal sequence). An exemplary TvoVMA S2 intein sequence is given by SEQ ID NO:26 (N terminal sequence) and SEQ ID NO:27 (C terminal sequence).

In some embodiments, fused proteins are separated via size exclusion chromatography or other appropriate methods known in the art. In certain embodiments configured to produce spider silk proteins or other fibrous proteins, the isolated high molecular weight proteins are then electrospun or otherwise drawn into fibers. These fibers can then be tested for strength, woven into desired applications, used in medical applications or any other suitable application. Similar processes may be utilized for other protein types and protein combinations described herein, including other fibrous proteins and combinations thereof described herein.

### Gene Editing Methods

Various gene editing methods may be utilized to target and modify the FibH gene of Bombyx mori. Most gene editing methods rely on targeted endonuclease activity and vary based on the particular endonuclease utilized and the corresponding targeting technique inherent to the endonuclease used. ZFNs or TALENs may be utilized but are typically less preferred due to the necessity of designing and constructing new for each target. Presently, more preferred methods include those that utilize clustered regularly interspaced short palindromic repeats (CRISPR) methods, including those that make use of the Mad7 nuclease or the Cas9 nuclease, for example.

The choice of gene editing process utilized to form the transgenic Bombyx mori affects the design of gRNAs and/or the particular portion of the FibH gene targeted for nuclease activity. That is, particular portions of the FibH gene targeted for knockout and/or as an insertion site will vary somewhat depending on the particular gene editing process utilized due to inherent differences in the target requirements and activity of the different nucleases (e.g., different PAM sequence requirements).

Although the particular examples of gRNAs and vectors described herein are designed for Cas9 and Mad7 systems, other gRNAs and/or target sites may be utilized where other gene editing systems are used. Although the particular target site of the FibH gene may vary with different systems and/or with different gRNAs in order to accommodate different nuclease functionality, the target should preferably still be within about 200 base pairs, more preferably about 100 base pairs, even more preferably within about 50 base pairs, of the target site when the disclosed gRNAs and their corresponding nuclease systems are utilized.

### Vector Construction

Vectors utilized to modify the FibH gene include one or more spider silk sequences and/or other sequences encoding for proteins of interest, such as one or more of the sequences provided by SEQ ID NO:9 to SEQ ID NO:13 and SEQ ID NO:16 to SEQ ID NO:19. The vectors may also include homology arms such as provided by SEQ ID NO:14 and SEQ ID NO:15.

The vectors also include homologous arms designed to guide insertion of the donor sequence(s) into the targeted portion of the FibH gene. The form of the homologous arms will vary depending on the particular site of the FibH gene targeted for nuclease activity. The homologous arms are designed to have sufficient homology to the remaining upstream and downstream portions of the Bombyx mori genome following nuclease activity in order to guide appropriate insertion via homology directed repair.

These exemplary homologous arm sequences may be varied somewhat, the downstream portion of the upstream homologous arm and the upstream portion of the downstream homologous arm in particular, to account for differences in FibH gene variants and/or different nuclease target sites as appropriate.

The use of a truncation vector (with minimal or no homology arms) versus an insertion vector involves different tradeoffs, and one may be preferred over another depending on particular application needs. For example, a truncation vector minimizes relatively more of the native silk encoding sequences.

The donor sequences described herein are much larger than donor sequences utilized in conventional Bombyx mori vectors. For example, a vector may include a donor sequence portion (i.e., the portion not including homologous arms) of greater than about 2 kbp, or greater than about 4 kbp, or greater than about 6 kbp, or greater than about 8 kbp, or greater than about 10 kbp, or greater than about 12 kbp, or greater than about 14 kbp, greater than about 16 kbp, or greater than about 18 kbp. A donor sequence may therefore range in size from about 2 kbp to about 20 kbp, though other ranges utilizing any two of the foregoing values as endpoints may also be utilized.

The large relative size of the donor sequence portion allows for a large resulting silk protein and the concomitant benefits to mechanical properties associated therewith. The size of the donor sequence portion may also be large relative to the homologous arms used to guide insertion, and yet still be able to be successfully introduced into the FibH gene. The homology arms are typically about 500 bp to 1 kbp, for example. Typically, the donor sequence insert is approximately the same size as the homology arms. Here, however, the disclosed vectors proved effective even though the donor sequence portion can be more than 2 times the size of the average size of the homology arms. More typically, the donor sequence is more than 5 times, more than 8 times, more than 12 times, more than 16 times, more than 20 times, more than 24 times, more than 28 times, more than 32 times, more than 36 times, or more than 40 times the average size of the homology arms. A donor sequence may therefore be about 2 to about 40 times the average size of the corresponding homology arms. For example, an insert of about 20 kbp may be paired with homology arms of about 500 bp. Other ranges utilizing any two of the foregoing values as endpoints may also be utilized.

In some embodiments, because two separate vectors are utilized in the split intein system, vectors may be provided that separately encode strength and elasticity domains of fibrous proteins (e.g., spider silk). For example, depending on the desired properties of the final fiber, a vector could be linked to an elasticity domain, or to two strength domains, or to two elasticity domains. Other combinations of strength and elasticity domains may be utilized to achieve desired overall properties of the final fused protein.

### Plasmid Construction & Cell Transformation

The vectors described herein may be inserted into plasmids. The plasmids may include features known in the art for enabling cloning and amplification. The plasmid may include an origin of replication, a suitable site for cloning (e.g., a multiple cloning site), a selection gene (e.g., ampicillin resistance), various regulatory sequences (e.g., promoters, binding sites, lac promoter and operon, etc.), and primer sites, for example. Various plasmid backbones are known in the art and are suitable for use with the vectors described herein. Examples include pUC57 and other plasmids of similar function and ability to receive vectors with the sizes disclosed herein.

In some embodiments, a plasmid can include the vector sequence as well as a sequence encoding for the nuclease (e.g., Cas9 or Mad7) of the associated gene editing process intended for incorporating the vector into the FibH gene. However, presently preferred embodiments deliver the nuclease and corresponding gRNAs separately in order to improve delivery and incorporation into the genome by preventing the plasmids from becoming too large.

Plasmids may be delivered to the target Bombyx mori cells via one or more suitable transformation methods known in the art. For example, silkworm eggs may be transformed via microinjection, electroporation, other transformation method, or combinations thereof. Following transformation, the plasmids may be linearized using targeted restriction enzymes and/or other known methods. In some embodiments, a nuclease target site associated with the nuclease of the corresponding gene editing method may be cloned into the plasmid such that the plasmid is itself targeted and linearized by the same nuclease used to target the host genome.

Introduction of the plasmids into the silkworm egg should preferably be done as soon as possible upon oviposition of the eggs. To aid in identifying possible transgenic eggs, a fluorescent marker such as GFP, dsRED, YFP or any other similar colorimetric protein can be linked to a silkworm egg stage promoter such as actin A3, Nos, 3xP3, or any other similar promoter. This promoter-colorimetric protein can then be integrated into a neutral site of the silkworm genome to ensure heritability. As described herein, incorporation to the neutral site can be done with any of the commonly used genetic engineering methods including but not limited to CRISPR, TALENs or Mad7.

In one embodiment, eggs that display the desired marker are then allowed to hatch, and silkworm from the GO generation are interbred. Eggs from the F1 generation are then screened once again via the appropriate fluorescent method. F1 eggs that have the fluorescent marker are separated and tracked throughout their lifecycle. Molts from individual silkworms are collected, DNA is extracted, and genetic markers and/or insertions or deletions are screened for via PCR or other sequencing methods.

Protein samples can be extracted from the silkworm silk gland during 5th instar and subjected to mass spectrometry, western blotting, or other forms of protein verification. Upon establishment of a consistent protein of interest producing strain, scaled up protein production can begin. Protein production through silkworms can involve numerous approaches, including but not limited to allowing the silkworm to naturally secrete the protein during cocoon spinning steps or excising silk glands from the 5th instar silkworms. Following collection of the proteins, cocoons are dissolved according to best practices, and the proteins affinity purified. In the case of the silk gland extraction, the glands are dissolved, and proteins of interest are purified.

Other organisms may be similarly transformed to provide for other types of protein production systems. Examples include microbial protein production systems based on E. coli, Bacillus, Actinomycetes, Pseudomonas, or lactic acid bacteria. Other microbial protein production systems include those based on yeast, including strains such as Saccharomyces cerevisiae, Pichia pastoris, or Schizosaccharomyces pombe. Transformation of these organisms may similarly be accomplished via microinjection, electroporation, other transformation methods, or combinations thereof.

Microbe-based protein production systems traditionally have difficulty producing large proteins and other proteins derived from repetitive DNA sequences. Because of this, the ability to use the existing E. coli or yeast-based protein production methods is limited when it comes to producing spider silk proteins or other proteins of similar size and complexity. Split intein reactions used in conjunction with microbe-based protein production systems help to overcome such limitations in these systems.

In some embodiments, transformation of a microbial host may be accomplished using plasmids comprising an appropriate origin of replication (ORI) (e.g., for E. coli or yeast), antibiotic resistance gene for selection, a strong inducible protein promoter, a protein purification tag such as his tag, the split intein sequence, and finally the desired protein sequence. The exact sequences for all of the above components can be varied depending on the needs of the production requirements.

In some embodiments, upon the successful transformation of protein production optimized microbial strains (e.g., E. coli such as BL-21 or yeast), and small-scale protein induction tests to ensure that the microbes are producing the desired protein, production of the protein can begin in accordance with known industry practices for microbial host protein production. For example, the synthesized proteins may be purified according to respective affinity tags, the split intein reaction is then allowed to proceed, the ligated protein is isolated via size exclusion chromatography or other appropriate isolation method known in the art, and the protein is further processed as desired (e.g., electrospun or otherwise drawn into fibers where applicable).

### Abbreviated List of Defined Terms

With respect to various terms of art and molecular biology details disclosed herein, reference is made to Sambrook, Fritsch, Maniatis, Molecular Cloning, A LABORATORY MANUAL (2d Edition, Cold Spring Harbor Laboratory Press, 1989) (especially Volume 3), and Kendrew, THE ENCYCLOPEDIA OF MOLECULAR BIOLOGY (Blackwell Science Ltd 1995). When combined with the teachings of this disclosure, the teachings of these references can be suitably modified, without undue experimentation, to enable the skilled artisan to utilize molecular biology techniques to construct the various vectors disclosed herein, to clone vectors into suitable plasmids, and to transform and form recombinant organisms (e.g., E. coli, yeast, Bombyx mori) useful for generating high molecular weight proteins of interest.

As used herein, a “non-native” protein is a protein that is not natively produced by standard (not artificially genetically modified) forms of the producing organism. For example, spider proteins or human proteins are non-native with respect to a Bombyx mori protein production system.

As used herein, a “high molecular weight protein” capable of being produced using the disclosed split intein systems is a protein with a molecular weight of at least about 340 kDa, or at least about 400 kDa, or at least about 450 kDa, or at least about 500 kDa, or at least about 550 kDa, or at least about 600 kDa, or at least about 650 kDa, or at least about 700 kDa, or at least about 750 kDa, or at least about 800 kDa, or has a size within a range with endpoints defined by any two of the foregoing values.

As used herein, “modification” of the fibroin heavy chain gene includes embodiments where the entire FibH gene, or one or more portions thereof, are knocked out of the silkworm genome and replaced with a knock-in insert (e.g., using a truncation vector). The term also includes embodiments where one or more inserts are inserted at a position within the FibH gene and/or at a position functionally adjacent to the gene (e.g., using an insertion vector).

The terms “donor sequence”, “donor sequence portion”, “donor portion”, “donor insert”, and related terms are used herein to refer to the portion of a vector not including the homology arms intended to guide insertion of the vector to the target site within the FibH gene. The donor sequence may include an NTD and/or CTD sequence in addition to one or more protein encoding sequences. Alternatively, the donor sequence may omit the NTD and CTD sequences. The terms “vector”, “insertion vector”, and the like are used to refer to the full sequence that includes the donor sequence (or multiple combined donor sequences) and the upstream and downstream homology arms.

The terms “homologous arms” and “homology arms” are used interchangeably herein to refer to the portion of the vector intended to be homologous to a corresponding portion of the native gene on each side of the targeted location where introduction of the donor sequence is intended. Depending on where the FibH gene is targeted for nuclease activity, the NTD and CTD of the vector (if included) can act in whole or in part as homology arms.

It should be understood that the proteins and the nucleic acids encoding them may differ from the exact sequences illustrated and described herein. Thus, this disclosure includes related sequences with deletions, additions, truncations, and substitutions to the sequences shown, so long as the sequences function in accordance with the methods of the invention. Accordingly, nucleotide sequences encoding functionally equivalent variants of the illustrated sequences and proteins are included in this disclosure. For instance, changes in a DNA sequence that do not change the encoded amino acid sequence, as well as those that result in conservative substitutions of amino acid residues, one or a few amino acid deletions or additions, and/or substitution of amino acid residues by amino acid analogs are those which will not significantly affect properties of the encoded polypeptide.

Conservative amino acid substitutions include glycine/alanine; valine/isoleucine/leucine; asparagine/glutamine; aspartic acid/glutamic acid; serine/threonine/methionine; lysine/arginine; and phenylalanine/tyrosine/tryptophan. Amino acids are generally divided into four families: (1) acidic-aspartate and glutamate; (2) basic-lysine, arginine, histidine; (3) non-polar-alanine, valine, leucine, isoleucine, proline, phenylalanine, methionine, tryptophan; and (4) uncharged polar-glycine, asparagine, glutamine, cysteine, serine threonine, tyrosine. Phenylalanine, tryptophan, and tyrosine are sometimes classified as aromatic amino acids. It is reasonably predictable that an isolated replacement of a leucine with an isoleucine or valine, or vice versa; an aspartate with a glutamate or vice versa; a threonine with a serine or vice versa; or a similar conservative replacement of an amino acid with a structurally related amino acid, will typically not have a major effect on activity and function of the overall protein. Proteins having substantially the same amino acid sequence as the sequences illustrated and described but possessing minor amino acid substitutions that do not substantially affect the activity or function of the protein are, therefore, within the scope of this disclosure.

Nucleotide sequences that have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the disclosed sequences may be considered functional equivalents.

Sequence identity or homology may be determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877. Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448. Advantageous for use according to the present invention is the WU-BLAST (Washington University BLAST) version 2.0 software. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877).

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A method of producing transgenic Bombyx mori, the method comprising:
providing a first vector having a first donor sequence that encodes a first non-native protein and at least one split intein domain;
providing a second vector having a second donor sequence that encodes a second non-native protein and at least one split intein domain;
incorporating the first vector into one or more Bombyx mori cells; and
incorporating the second vector into one or more Bombyx mori cells,
wherein the split intein domains encoded by the first and second vectors are configured to associate with one another and ligate the first and second non-native proteins to thereby form a fused protein.

2. The method of claim 1, further comprising providing a gene editing assembly that includes a nuclease configured to target one or more locations within a silk protein gene of the Bombyx mori.

3. The method of claim 2, wherein the gene editing assembly targets the FibH gene.

4. The method of claim 1, wherein the first donor sequence, the second donor sequence, or both encode for a spider silk protein.

5. The method of claim 4, wherein the spider silk protein comprises an AS28 protein, a MaSp1 protein, a MaSp4 protein, or combination thereof.

6. The method of claim 4, wherein the spider silk protein is associated with an orb-weaver spider.

7. The method of claim 6, wherein the orb-weaver spider is Caerostris darwini.

8. The method of claim 1, wherein the first donor sequence and the second donor sequence encode different spider silk proteins.

9. The method of claim 1, wherein the first donor sequence and the second donor sequence both encode a scleroprotein.

10. The method of claim 9, wherein the first donor sequence, the second donor sequence, or both encode a collagen, elastin, keratin, or fibrin.

11. The method of claim 10, wherein the first donor sequence, the second donor sequence, or both encode a human protein.

12. The method of claim 1, further comprising providing a third vector having a third donor sequence that encodes for a third non-native protein and at least one split intein domain,
wherein the second donor sequence encodes two split intein domains, one on each terminal of the second non-native protein,
wherein one split intein domain of the second non-native protein functions to ligate the second non-native protein to the first non-native protein, and
wherein the other split intein domain of the second non-native protein functions to ligate the second non-native protein to the third non-native protein.

13. The method of claim 12, further comprising providing a fourth vector having a fourth donor sequence that encodes for a fourth non-native protein and at least one split intein domain,
wherein the third donor sequence encodes two split intein domains, one on each terminal of the third non-native protein,
wherein one split intein domain of the third non-native protein functions to ligate the third non-native protein to the second non-native protein, and
wherein the other split intein domain of the third non-native protein functions to ligate the third non-native protein to the fourth non-native protein.

14. A transgenic Bombyx mori silkworm made according to the method of claim 1.

15. A protein produced by the transgenic Bombyx mori silkworm of claim 14.

16. The protein of claim 15, wherein the protein includes spider silk.

17. The protein of claim 16, wherein the protein additionally includes a human scleroprotein.

18. The protein of claim 17, wherein the protein includes:
an A2S8 protein, a MaSp1 protein, a MaSp4 protein, or combination thereof, and
human collagen, human elastin, human keratin, human fibrin, or combination thereof.

19. A set of vectors for use in a Bombyx mori split intein system enabling the production of fused proteins from multiple non-native proteins, the set of vectors comprising:
a first vector having a first donor sequence that encodes (i) a first non-native protein and (ii) at least one split intein domain;
a second vector having a second donor sequence that encodes (i) a second non-native protein and (ii) at least one split intein domain,
wherein the split intein domains encoded by the first and second vectors are configured to associate with one another and ligate the first and second non-native proteins to thereby form a fused protein.

20. A method of producing a transgenic microbe, the method comprising:
providing a first vector having a first donor sequence that encodes a first non-native protein and at least one split intein domain;
providing a second vector having a second donor sequence that encodes a second non-native protein and at least one split intein domain;
incorporating the first vector into one or more microbes; and
incorporating the second vector into one or more microbes,
wherein the split intein domains encoded by the first and second vectors are configured to associate with one another and ligate the first and second non-native proteins to form a fused protein.
